(19) United States
(12) Patent Application Publication
(10) Pub. No.: US 2014/0065075 A1
(43) Pub. Date: Mar. 6, 2014

(54) TARGETED CONTRAST AGENTS AND USES THEREOF

(71) Applicants: Jidong Zhang, Ottawa (CA); Kenneth Curry, Richmond (CA)

(72) Inventors: Jidong Zhang, Ottawa (CA); Kenneth Curry, Richmond (CA)

(73) Assignee: RF THERAPEUTICS INC., Richmond, BC (CA)

(21) Appl. No.: 14/112,349

(22) PCT Filed: Apr. 17, 2012
(86) PCT No.: PCT/CA2012/000373

§ 371 Date: Nov. 20, 2013

Related U.S. Application Data
(60) Provisional application No. 61/477,288, filed on Apr. 20, 2011.

(51) Int. Cl. A61K 49/10; A61K 49/08

(52) U.S. Cl. CPC A61K 49/108; A61K 49/085; A61K 49/105
USPC 424/9.363; 540/465; 562/565; 424/9.365

(57) ABSTRACT

Described herein is a contrast agent for administration to a subject. The contrast agent includes a targeting portion that includes an unchelated aminocarboxylate functional group; a metal ion bound to a metal-complexable portion; and a linker joining the targeting portion and the metal-complexable portion of the contrast agent. The portion that is not bound to a metal ion is for binding to necrotic tissue in the subject.

### CROSS REFERENCE TO RELATED APPLICATIONS

This application claims the benefit of priority of U.S. Provisional Patent Application No. 61/477,288 filed Apr. 20, 2011, which is incorporated herein by reference in its entirety.

### FIELD

The present disclosure relates generally to novel contrast agents and their methods of use.

### BACKGROUND

Medical diagnostic imaging has evolved as an important non-invasive tool for medical diagnosis. Nuclear magnetic resonance imaging (“MRI”) and computerized tomography (“CT”) are two of the most widely used imaging methods. MRI generally relies on the relaxation properties of excited hydrogen nuclei in water. When the tissues or organs to be imaged are placed in a powerful, uniform magnetic field, the spins of the hydrogen protons within the tissues or organs align along the axis of the magnetic field. Medical imaging technologies also include ultrasound, SPECT or positron emission technology (PET) scans.

Imaging diagnosis plays an important role in medicine because it facilitates the accurate localization and characterization of disease that is critical for therapeutic decision-making and for the overall outcome of patient management. Due to technical innovations, imaging technologies have become much more powerful and versatile.

Although diagnostic imaging may be performed without the administration of contrast agents, the ability to improve the visualization of internal structures, for example tissues and organs, and fluids has resulted in the widespread use of contrast agents. Contrast agents are used to highlight specific areas to increase the visibility of the area being studied. Contrast agents for MRI technology alter the relaxation times of tissues and body cavities where they are located and work by shortening the relaxation time of protons located nearby.

The use of injectable contrast agents in conjunction with imaging techniques has increased dramatically over the last decade. These currently used contrast agents are generally safe, however, they are associated with some undesirable side effects. These side effects are divided into four major areas: systemic reactions, cardiac effects, renal effects, and general vascular effects. There have been many attempts to develop new contrast agents, with a primary aim of lessening the adverse effects.

Despite improvements in spatial and temporal resolution of diagnostic imaging, it remains difficult to make an unambiguous diagnosis even with the use of contrast agents. This problem may be attributed to the fact that there is substantial overlap in imaging signals between both pathological and normal tissues. One approach to solve this problem is to develop more specific contrast agents that specifically concentrate in targeted organs or tissues.

The use of porphyrins over the past decades sparked an interest in the development of new compounds that exhibit targeting capabilities. However, problems related to many porphyrin based contrast agents include instability, discoloration, toxicity and slow clearance rates. Several patent applications such as WO 00/09169 and WO 02/38546 discuss various non-porphyrin contrast agents that exhibit some “targeting” abilities however, problems related the reproducibility of these compounds along with slow clearance rates and longevity of the compound within the patient continue to exist.

It is, therefore, desirable to provide a new class of contrast agent compounds which aim to develop a new targeted contrast agent having the desired pharmacokinetic related clearance properties and minimized toxicity and/or side-effects.

### SUMMARY

It is an object of the present disclosure to obviate or mitigate at least one disadvantage of previous contrast agents.

In a first aspect, the present disclosure is based, in part, on the unexpected discovery that a targeting portion including an aminocarboxylate functional group is able to target and bind necrotic tissue.

The present disclosure provides a novel class of contrast agents comprising a targeting portion including an unchelated aminocarboxylate functional group, a metal ion bound to a metal-complexable portion, and a linker joining the targeting portion and the metal-complexable portion of the contrast agent, wherein the portion that is not bound to a metal ion, on administration of the chelating agent to a subject, binds to necrotic tissue.

In some embodiments, the metal complexable portion of the contrast agent includes an aminocarboxylate functional group.

In some embodiments, the aminocarboxylate functional group of the contrast agent is a polyaminocarboxylate functional group.

In some embodiments, the targeting portion of the contrast agent is capable of complexing a metal, wherein only one metal ion is bound to the contrast agent and the metal ion and the contrast agent are in a 1:1 molar ratio and wherein one of the two portions of the contrast agent includes an unchelated aminocarboxylate functional group.

In some embodiments, there is provided a contrast agent comprising the structure X-L-Y*M, wherein X is the targeting portion, L is the linker, and Y*M is the metal ion (M) bound to the metal-complexable portion (Y) of the contrast agent and wherein only one metal ion is bound to the contrast agent and the metal ion and the contrast agent are in a 1:1 molar ratio.

In some embodiments, the contrast agents of the present disclosure are useful as therapeutic agents and/or diagnostic agents.

In some embodiments, the contrast agents of the present disclosure may be useful in medical applications involving necrosis and necrosis-related pathologies.

In some embodiments, the contrast agents of the present disclosure are useful for the manufacture of compounds and/or medicaments suitable for use in diagnostic imaging or imaging-aided applications, including for example MRI, CT, SPECT, PET, MRI-aided applications, CT-aided applications, SPECT-aided applications, or PET-aided applications.

In some embodiments, the contrast agents of the present disclosure are provided in combination with pharmaceutically acceptable carriers.

In some embodiments, the contrast agents of the present disclosure may be useful to monitor the effectiveness of an ongoing therapeutic treatment.

Other aspects and features of the present disclosure will become apparent to those ordinarily skilled in the art upon review of the following description of specific embodiments in conjunction with the accompanying figures.

### BRIEF DESCRIPTION OF THE DRAWINGS

Embodiments of the present disclosure will now be described, by way of example only, with reference to the attached Figures.

FIG. 1 is a schematic representation showing the synthesis of RF1002;

FIG. 2 is a schematic representation showing the synthesis of RF1003;

FIG. 3 is a schematic representation showing the synthesis of RF1004;

FIG. 4 is a schematic representation showing the synthesis of RF1005;

FIG. 5 is a schematic representation showing the synthesis of RF1006;

FIG. 6 is a schematic representation showing the synthesis of RF1101;

FIG. 7 is a schematic representation showing the synthesis of RF1102;

FIG. 8 is a schematic representation showing the synthesis of RF1103;

FIG. 9 is a schematic representation showing the synthesis of RF1104;

FIG. 10 is a schematic representation showing the synthesis of RF1105;

FIG. 11 is a schematic representation showing the synthesis of RF1107;

FIG. 12 is a schematic representation showing the synthesis of RF1201;

FIG. 13 is a schematic representation showing the synthesis of RF1202;

FIG. 14 is a schematic representation showing the synthesis of RF1203;

FIG. 15 are magnetic resonance images of a control animal (A) and animals (B) through (D) following administration of contrast agent RF 1002 at a dosage of 5 mg/kg and corresponding tissue samples showing the degree of necrotic tissue;

FIG. 16 are magnetic resonance images of a control animal (A) and animals (B) through (D) following administration of contrast agent RF 1002 at a dosage of 40 mg/kg and corresponding tissue samples showing the degree of necrotic tissue;

FIG. 17 are magnetic resonance images of a control animal (A) and animals (B) through (D) following administration of contrast agent RF 1003 at a dosage of 5 mg/kg and corresponding tissue samples showing the degree of necrotic tissue;

FIG. 18 are magnetic resonance images of a control animal (A) and animals (B) through (D) following administration of contrast agent RF 1003 at a dosage of 40 mg/kg and corresponding tissue samples showing the degree of necrotic tissue;

FIG. 19 are magnetic resonance images of a control animal (A) and animals (B) through (D) following administration of contrast agent RF 1004 at a dosage of 5 mg/kg and corresponding tissue samples showing the degree of necrotic tissue; and

FIG. 20 are magnetic resonance images of a control animal (A) and animals (B) through (D) following administration of contrast agent RF 1004 at a dosage of 40 mg/kg and corresponding tissue samples showing the degree of necrotic tissue.

### DETAILED DESCRIPTION

Generally, the present disclosure provides a novel class of contrast agents comprising, a targeting portion including an unchelated aminocarboxylate functional group a metal ion bound to a metal-complexable portion, and a linker joining the targeting portion and the metal-complexable portion of the contrast agent. The portion that is not bound to a metal ion, on administration of the chelating agent to a subject, binds to necrotic tissue.

Traditional contrast agents have chemical structures where a single chelating agent is bound to a metal ion to form a complex such as Magnevist® (Gd-DTPA), Dotarem® (Gd-DOTA), Omniscan® (Gd-DTPA-BMA) and ProHance® (Gd-HPDO3A). None of these traditional agents target specific organs or tissue of interest and often they are associated with unfavorable pharmacokinetics. These types of non-specific traditional agents typically have a very short half-life in plasma and a short time window for contrast-enhanced imaging which make it difficult to estimate the optimal imaging timing.

As used herein, the term ‘subject’ refers to an animal, such as a bird or a mammal. Specific animals include rat, mouse, dog, cat, cow, sheep, horse, pig or primate. A subject may further be a human, alternatively referred to as a patient. A subject may further be a transgenic animal. A subject may further be a rodent, such as a mouse or a rat.

Chelation is commonly applied in many areas, for example metal complex chemistry, organic and inorganic chemistry, and biochemistry. Chelating agents are used to control metal ions in aqueous systems, thus their popularity in the area of contrast agents in binding metal ions for use in diagnostic imaging. Chelating agents form stable water soluble complexes with multivalent metal ions and prevent undesired interaction by blocking normal reactivity of metal ions. Contrast agents of the present disclosure are T1 relaxation agents comprising a metal ion that is bound into a chelate complex. The MRI signal intensity relates to the value of the relaxation rate of tissues.

In general, the relaxation efficiency of a T1 contrast agent depends on several factors, including the nature of the metal ion and size and structure of the metal-chelate complex. T1 relaxation agents act as a relaxation sink for water protons. Paramagnetic metal chelates, for example, Gd(III), Fe(III), and Mn(II) complexes, may alter the relaxation rate of the surrounding water protons to allow for more effective MRI contrast enhancement. Chelate molecules are relatively large and have many bonds with the metal ion. There is a limited amount of free space within layer of atoms surrounding the metal ion, known as the coordination sphere. This lack of free space generally prevents the protons of the larger chelate molecule from getting sufficiently close to the metal ion for efficient energy transfer. As a result the tissue water is able to diffuse into the coordination sphere of the metal ion and give up its energy, and then exchange with the tissue water in turn enabling additional water molecules to enter the coordination sphere. The diffusion exchange occurs very quickly and the result is that the tissue water near the contrast agent has a larger net magnetization than the water in the neighboring tissue and contributes a stronger signal in a T1-weighted image.

The surprising discovery of the ability of a targeting portion including an aminocarboxylate functional group to target and bind necrotic tissue has led to the development of the novel class of contrast agents disclosed herein.

These novel contrast agents comprise: a targeting portion including an unchelated aminocarboxylate functional group, a metal ion bound to a metal-complexable portion, and a linker joining the targeting portion and the metal-complexable portion. A person of skill in the art would understand a metal ion bound to a metal-complexable portion may also be referred to as a metal chelate. The targeting portion is free to bind to necrotic tissue following administration of a contrast agent to a subject.

In some embodiments, the contrast agents may be represented by the formula: X-L-Y*M, where X is the targeting portion, L is the linker, and Y*M is the metal ion (M) bound to the metal-complexable portion (Y). As illustrated by this formula, only one metal ion is bound to the contrast agent and the metal ion and the contrast agent are in a 1:1 molar ratio.

In some embodiments, the contrast agents may be represented by the formula: X-L-(Y*M)₂, where X is the targeting portion, L is the linker, and (Y*M)₂ represents two metal ions (M) bound to two metal-complexable portions (Y). As illustrated by this formula, there are two metal ions bound to the contrast agent and the metal ion and the contrast agent are in a 2:1 molar ratio and the targeting portion is free to bind to necrotic tissue following administration of a contrast agent to a subject. Contrast agents having (Y*M)ₙ where n is 2, 3, 4 or 5 are also contemplated where the targeting portion is free to bind to necrotic tissue following administration of a contrast agent to a subject.

In some embodiment, the aminocarboxylate functional group has between 1 and 10 carboxylate groups, preferably between 1 and 9 carboxylate groups, preferably between 1 and 8 carboxylate groups, preferably between 1 and 7 carboxylate groups, preferably between 1 and 6 carboxylate groups, preferably between 1 and 5 carboxylate groups, preferably between 1 and 4 carboxylate groups, preferably between 1 and 3 carboxylate groups, and preferably between 1 and 2 carboxylate groups. In an alternative embodiment, the aminocarboxylate functional group has between 2 and 4 carboxylate groups. Each carboxylate group may be coordinated with a cation, for example H⁺, Na⁺, K⁺, or any other cation which allows the carboxylate group to bind to necrotic tissue.

In some embodiments, the aminocarboxylate functional group has between 1 and 10 amino groups, preferably between 1 and 9 amino groups, preferably between 1 and 8 amino groups, preferably between 1 and 7 amino groups, preferably between 1 and 6 amino groups, preferably between 1 and 5 amino groups, preferably between 1 and 4 amino groups, preferably between 1 and 3 amino groups, and preferably between 1 and 2 amino groups. In an alternative embodiment, the aminocarboxylate functional group has between 1 and 3 amino groups.

In some embodiments, the aminocarboxylate functional group has between 4 and 50 carbon atoms, preferably between 4 and 46, preferably between 4 and 42, preferably between 4 and 38, preferably between 4 and 34, preferably between 4 and 30, preferably between 4 and 26, preferably between 4 and 22, preferably between 4 and 18, preferably between 4 and 14, preferably between 4 and 10 and preferably between 4 and 6. In an alternative embodiment, the aminocarboxylate functional group has between 6 and 14 carbon atoms, preferably between 6 and 10, preferably between 10 and 14.

In some embodiments, the aminocarboxylate functional group has a molecular weight between about 100 and 1000 atomic mass units, preferably about 100 to about 900, preferably about 100 to about 800, preferably about 100 to about 700, preferably about 100 to about 600, preferably about 100 to about 500, preferably about 100 to about 400, preferably about 100 to about 300 and preferably about 100 to about 200 atomic mass units. In an alternative embodiment, the aminocarboxylate functional group has a molecular weight between 125 to about 400 atomic mass units.

In some embodiments, the metal complexable portion may be an aminocarboxylate functional group. In one aspect, the aminocarboxylate functional group may be a polyaminocarboxylate functional group.

In some embodiments, the targeting portion may be capable of complexing a metal. In such embodiments, it would be understood that only one metal ion is bound to the contrast agent and the metal ion and the contrast agent are in a 1:1 molar ratio, and that one of the two portions includes an unchelated aminocarboxylate functional group.

In some embodiments, X and Y may be the same metal complexable portions, for example X-L-Y could be represented by Y-L-Y. In such embodiments, it should be understood that only one metal ion is bound to the contrast agent and the metal ion and the contrast agent are in a 1:1 molar ratio, and that one of the two portions includes an unchelated aminocarboxylate functional group.

### DEFINITIONS

The term “linker” as used herein denotes a bond or chemical group that joins two or more other chemical groups. For example, in joining chemical groups R and R′, a linker may be a bond that links R and R′ directly, or may be a chemical group that is linked to R and R′ via, for example, amide, ester, ether, hydrazide, nitrogen, or sulfur functionalities.

The linker may be alkyl, heteroalkyl, alkoxy, alkoxyalkyl, acyl, cycloalkyl, cycloalkylalkyl, aryl, heteroaryl, heterocycloalkyl, hydroxyalkyl, alkylthio, alkylcarbonylamino, alkylsulfinyl, alkylsulfonyl, alkylsulfonylamino, or heteroalkoxy. Preferably, the linker is an alkyl, aryl, heteroalkyl or heteroaryl linker.

Specific examples of linker groups include, but are not limited to, R—R′, R—NH—C₆H₄—NH—R′, R—NH—C₆H₈—NH—R′, R—CH₂CH₂—R′, R—NHCH₂CH₂NH—R′, R—NHNH—R′, R—NH—R′, R—O—R′, R—C(═O)—R′, R—NH—(C═O)—R′, R—NH—(C═O)—NH—R′, and R—NHNH—(C═O)—CH₂NH—R′, where R and R′ represent the two chemical groups being linked together.

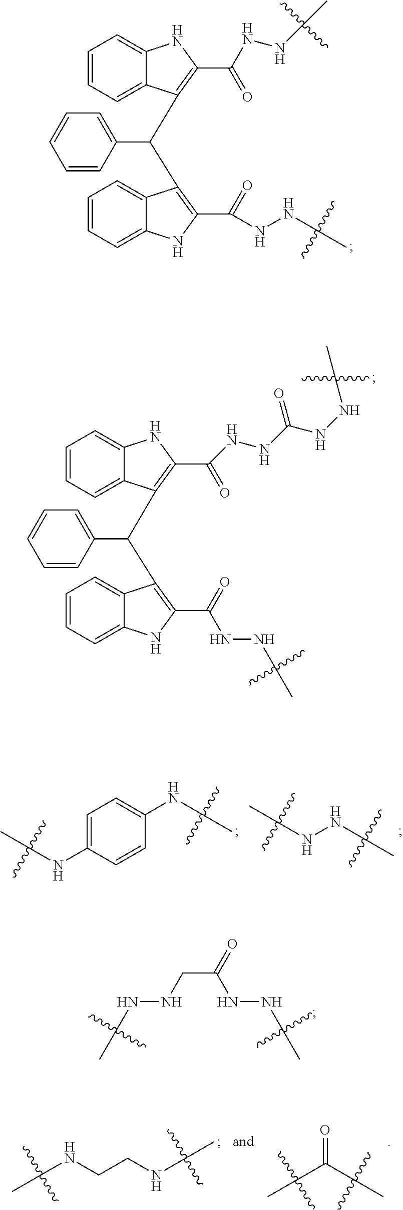

The term “aminocarboxylate portion” as used herein denotes a chemical group with at least one amino group and a plurality of carboxylate groups. In one embodiment, the aminocarboxylate portion is a chemical group with a plurality of amino groups and a plurality of carboxylate groups.

Specific examples of aminocarboxylate portion include:

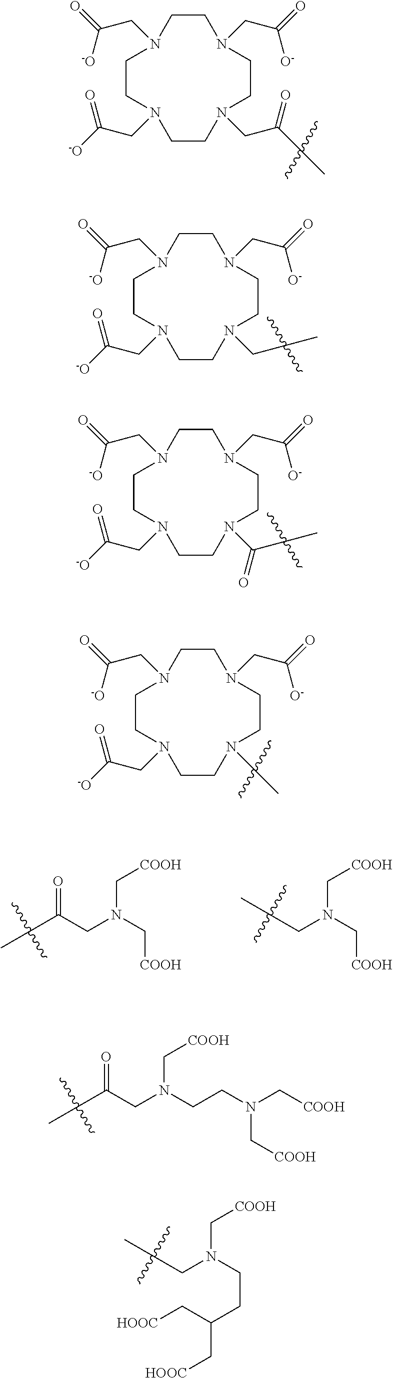

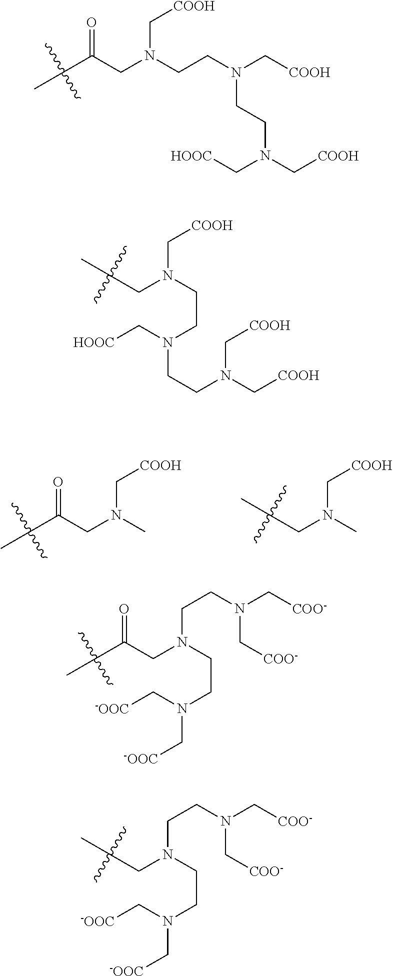

The term “metal-complexable portion” as used herein denotes a chemical group having ligands that can bond to a central metal atom to form a chelate complex. When acting as a magnetic resonance imaging (MRI) contrast agent, the chelate complex provides the metal with a coordination site to coordinate with a water molecule. The relaxation time of the complexed water molecule is altered and can be more readily discerned in an MRI image. Specific examples of metal-complexable portion include:

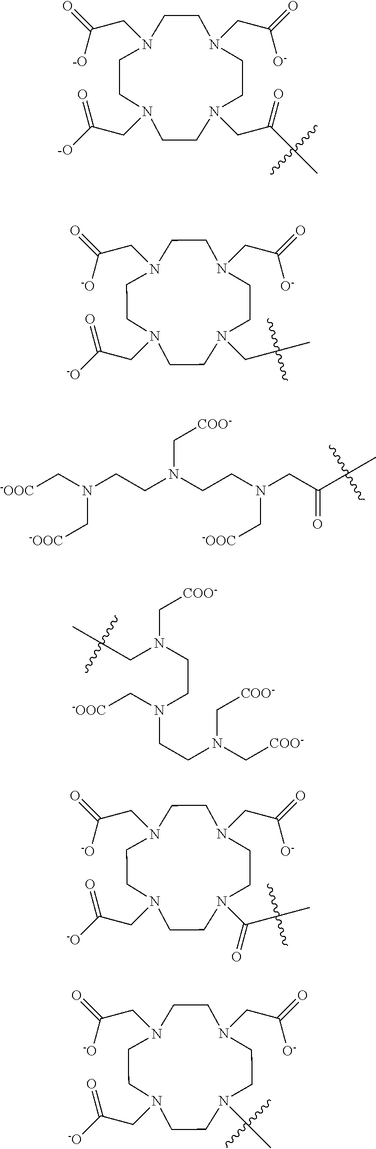

The term “alkyl” as used herein denotes an unbranched or branched chain, saturated, hydrocarbon residue containing 1 to 20 carbon atoms. The term “lower alkyl” denotes a straight or branched chain hydrocarbon residue containing 1 to 10 carbon atoms. “C₁₋₁₀ alkyl” as used herein refers to an alkyl composed of 1 to 10 carbons. Examples of alkyl groups include, but are not limited to, lower alkyl groups include methyl, ethyl, propyl, i-propyl, n-butyl, i-butyl, t-butyl or pentyl, isopentyl, neopentyl, hexyl, heptyl, and octyl.

When the term “alkyl” is used as a suffix following another term, as in “phenylalkyl,” or “hydroxyalkyl,” this is intended to refer to an alkyl group, as defined above, being substituted with one to two substituents selected from the other specifically-named group. Thus, for example, “phenylalkyl” denotes the radical R′R″—, wherein R′ is a phenyl radical, and R″ is an alkylene radical as defined herein with the understanding that the attachment point of the phenylalkyl moiety will be on the alkylene radical. Examples of arylalkyl radicals include, but are not limited to, benzyl, phenylethyl, 3-phenylpropyl. The terms “arylalkyl” or “aralkyl” are interpreted similarly except R′ is an aryl radical. The terms “(het)arylalkyl” or “(het)aralkyl” are interpreted similarly except R′ is optionally an aryl or a heteroaryl radical.

“Heteroalkyl” means an alkyl moiety as defined herein, including a branched alkyl, which includes one or more heteroatoms. Exemplary heteroalkyl moieties can have one, two or three hydrogen atoms be replaced with a substituent independently selected from the group consisting of —OR^{a}, —NR^{b}R^{c}, and —S(O)ₙR^{d} (where n is an integer from 0 to 2), wherein R^{a} is hydrogen, acyl, alkyl, cycloalkyl, or cycloalkylalkyl; R^{b} and R^{c} are independently of each other hydrogen, acyl, alkyl, cycloalkyl, or cycloalkylalkyl; and when n is 0, R^{d} is hydrogen, alkyl, cycloalkyl, or cycloalkylalkyl; when n is 1, R^{d} is alkyl, cycloalkyl, or cycloalkylalkyl; and when n is 2, R^{d} is alkyl, cycloalkyl, cycloalkylalkyl, amino, acylamino, monoalkylamino, or dialkylamino. Other heteroalkyl moieties can have one or more heteroatoms inserted between carbon atoms. Representative examples include, but are not limited to, 2-hydroxyethyl, 3-hydroxypropyl, 2-hydroxy-1-hydroxymethylethyl, 2,3-dihydroxypropyl, 1-hydroxymethylethyl, 3-hydroxybutyl, 2,3-dihydroxybutyl, 2-hydroxy-1-methylpropyl, 2-aminoethyl, 3-aminopropyl, 2-methylsulfonylethyl, aminosulfonylmethyl, aminosulfonylethyl, aminosulfonylpropyl, methylaminosulfonylmethyl, methylaminosulfonylethyl, methylaminosulfonylpropyl, methylethylether, dimethylamine, adipic acid dihydrazide, and the like.

The term “alkylene” as used herein denotes a divalent saturated linear hydrocarbon radical of 1 to 20 carbon atoms (e.g., (CH₂)ₙ) or a branched saturated divalent hydrocarbon radical of 2 to 20 carbon atoms (e.g., —CHMe— or —CH₂CH(i-Pr)CH₂—), unless otherwise indicated. Except in the case of methylene, the open valences of an alkylene group are not attached to the same atom. Examples of alkylene radicals include, but are not limited to, methylene, ethylene, propylene, 2-methyl-propylene, 1,1-dimethyl-ethylene, butylene, 2-ethylbutylene.

The term “alkoxy” as used herein means an —O-alkyl group, wherein alkyl is as defined above such as methoxy, ethoxy, n-propyloxy, i-propyloxy, n-butyloxy, i-butyloxy, t-butyloxy, pentyloxy, hexyloxy, including their isomers. “Lower alkoxy” as used herein denotes an alkoxy group with a “lower alkyl” group as previously defined. “C₁₋₁₀ alkoxy” as used herein refers to an —O-alkyl wherein alkyl is C₁₋₁₀.

The term “alkoxyalkyl” as used herein refers to the radical R′R″, wherein R′ is an alkoxy radical as defined herein, and R″ is an alkylene radical as defined herein with the understanding that the attachment point of the alkoxyalkyl moiety will be on the alkylene radical. C₁₋₆ alkoxyalkyl denotes a group wherein the alkyl portion is comprised of 1-6 carbon atoms exclusive of carbon atoms in the alkoxy portion of the group. C₁₋₃ alkoxy-C₁₋₆ alkyl denotes a group wherein the alkyl portion is comprised of 1-6 carbon atoms and the alkoxy group is 1-3 carbons. Examples are methoxymethyl, methoxyethyl, methoxypropyl, ethoxymethyl, ethoxyethyl, ethoxypropyl, propyloxypropyl, methoxybutyl, ethoxybutyl, propyloxybutyl, butyloxybutyl, t-butyloxybutyl, methoxypentyl, ethoxypentyl, propyloxypentyl including their isomers.

The term “acyl” as used herein denotes a group of formula —C(═O)R wherein R is hydrogen or lower alkyl as defined herein. The term or “alkylcarbonyl” as used herein denotes a group of formula C(═O)R wherein R is alkyl as defined herein. The term C₁₋₆ acyl refers to a group —C(═O)R contain 6 carbon atoms. The term “arylcarbonyl” as used herein means a group of formula C(═O)R wherein R is an aryl group; the term “benzoyl” as used herein an “arylcarbonyl” group wherein R is phenyl.

“Cycloalkyl” means a saturated carbocyclic moiety consisting of mono- or bicyclic rings. Cycloalkyl can optionally be substituted with one or more substituents, wherein each substituent is independently hydroxy, alkyl, alkoxy, halo, haloalkyl, amino, monoalkylamino, or dialkylamino, unless otherwise specifically indicated. Examples of cycloalkyl moieties include, but are not limited to, cyclopropyl, cyclobutyl, cyclopentyl, cyclohexyl, cycloheptyl, and the like, including partially unsaturated derivatives thereof.

“Cycloalkylalkyl” mean a moiety of the formula —R^{a}—R^{b}, where R^{a} is alkylene and R^{b} is cycloalkyl as defined herein.

“Aryl” means a cyclic aromatic hydrocarbon moiety consisting of a mono-, bi- or tricyclic aromatic ring. The aryl group can be optionally substituted as defined herein. Examples of aryl moieties include, but are not limited to, optionally substituted phenyl, naphthyl, phenanthryl, fluorenyl, indenyl, pentalenyl, azulenyl, oxydiphenyl, biphenyl, methylenediphenyl, aminodiphenyl, diphenylsulfidyl, diphenylsulfonyl, diphenylisopropylidenyl, benzodioxanyl, benzofuranyl, benzodioxylyl, benzopyranyl, benzoxazinyl, benzoxazinonyl, benzopiperadinyl, benzopiperazinyl, benzopyrrolidinyl, benzomorpholinyl, methylenedioxyphenyl, ethylenedioxyphenyl, and the like, including partially hydrogenated derivatives thereof.

The term “heteroaryl” or “heteroaromatic” as used herein means a monocyclic, bicyclic or tricyclic radical having at least one aromatic ring containing four to eight atoms per ring, incorporating one or more N, O, or S heteroatoms, the remaining ring atoms being carbon, with the understanding that the attachment point of the heteroaryl radical will be on an aromatic ring. As well known to those skilled in the art, heteroaryl rings have less aromatic character than their all-carbon counter parts. Thus, for the purposes of the application, a heteroaryl group need only have some degree of aromatic character. Examples of heteroaryl moieties include monocyclic aromatic heterocycles having 5 to 6 ring atoms and 1 to 3 heteroatoms include, but is not limited to, pyridinyl, pyrimidinyl, pyrazinyl, pyrrolyl, pyrazolyl, imidazolyl, oxazol, isoxazole, thiazole, isothiazole, triazoline, thiadiazole and oxadiaxoline which can optionally be substituted with one or more, preferably one or two substituents selected from hydroxy, cyano, alkyl, alkoxy, thio, lower haloalkoxy, alkylthio, halo, haloalkyl, alkylsulfinyl, alkylsulfonyl, halogen, amino, alkylamino, dialkylamino, aminoalkyl, alkylaminoalkyl, and dialkylaminoalkyl, nitro, alkoxycarbonyl and carbamoyl, alkylcarbamoyl, dialkylcarbamoyl, arylcarbamoyl, alkylcarbonylamino and arylcarbonylamino. Examples of bicyclic moieties include, but are not limited to, quinolinyl, isoquinolinyl, benzofuryl, benzothiophenyl, benzoxazole, benzisoxazole, benzothiazole and benzisothiazole. Bicyclic moieties can be optionally substituted on either ring; however the point of attachment is on a ring containing a heteroatom.

The term “heterocyclyl”, “heterocycle”, or “heterocycloalkyl” as used herein denotes a saturated cyclic radical, consisting of one or more rings, preferably one to two rings, of three to eight atoms per ring, incorporating one or more ring heteroatoms (chosen from N, O or S(O)₀₋₂), and which can optionally be independently substituted with one or more, preferably one or two substituents selected from hydroxy, oxo, cyano, lower alkyl, lower alkoxy, lower haloalkoxy, alkylthio, halo, haloalkyl, hydroxyalkyl, nitro, alkoxycarbonyl, amino, alkylamino, alkylsulfonyl, arylsulfonyl, alkylaminosulfonyl, arylaminosulfonyl, alkylsulfonylamino, arylsulfonylamino, alkylaminocarbonyl, arylaminocarbonyl, alkylcarbonylamino, arylcarbonylamino, unless otherwise indicated. Examples of heterocyclic radicals include, but are not limited to, azetidinyl, pyrrolidinyl, hexahydroazepinyl, oxetanyl, tetrahydrofuranyl, tetrahydrothiophenyl, oxazolidinyl, thiazolidinyl, isoxazolidinyl, morpholinyl, piperazinyl, piperidinyl, tetrahydropyranyl, thiomorpholinyl, quinuclidinyl and imidazolinyl. Preferrably “heterocyclyl”, “heterocycle”, or “heterocycloalkyl” is a morpholinyl, pyrrolidinyl, piperidinyl or tetrahydrofuranyl.

The term “hydroxyalkyl” as used herein denotes an alkyl radical as herein defined wherein one to three hydrogen atoms on different carbon atoms is/are replaced by hydroxyl groups.

The term “alkylthio” or “alkylsulfanyl” refers to an —S-alkyl group, wherein alkyl is as defined above such as meththio, ethylthio, n-propylthio, i-propylthio, n-butylthio, hexylthio, including their isomers. “Lower alkylthio” as used herein denotes an alkylthio group with a “lower alkyl” group as previously defined. “C₁₋₁₀ alkylthio” as used herein refers to an —S-alkyl wherein alkyl is C₁₋₁₀. “Phenylthio” is an “arylthio” moiety wherein aryl is phenyl.

The terms “alkylcarbonylamino” and “arylcarbonylamino” as used herein refers to a group of formula —NC(═O)R wherein R is alkyl or aryl respectively and alkyl and aryl are as defined herein.

The terms “alkylsulfinyl” and “arylsulfinyl” as used herein refers to a group of formula —S(═O)R wherein R is alkyl or aryl respectively and alkyl and aryl are as defined herein

The terms “alkylsulfonyl” and “arylsulfonyl” as used herein refers to a group of formula —S(═O)₂R wherein R is alkyl or aryl respectively and alkyl and aryl are as defined herein. The term “heteroalkylsulfonyl” as used herein refers herein denotes a group of formula —S(═O)₂R wherein R is “heteroalkyl” as defined herein.

The terms “alkylsulfonylamino” and “arylsulfonylamino” as used herein refers to a group of formula —NR′S(═O)₂R wherein R is alkyl or aryl respectively, R′ is hydrogen or C₁₋₃ alkyl, and alkyl and aryl are as defined herein.

The term “heteroalkoxy” as used herein means an —O-(heteroalkyl) group wherein heteroalkyl is defined herein. “C₁₋₁₀ heteroalkoxy” as used herein refers to an —O-(heteroalkyl) wherein alkyl is C₁₋₁₀. Representative examples include, but are not limited to, 2-dimethylaminoethoxy and 3-sulfonamido-1-propoxy.

The terms “halo,” “halogen,” and “halide” are used interchangeably herein and refer to fluoro, chloro, bromo, and iodo. “Haloalkyl” means alkyl as defined herein in which one or more hydrogen has been replaced with same or different halogen. Exemplary haloalkyls include —CH₂Cl, —CH₂CF₃, —CH₂CCl₃, —CF₂CF₃, —CF₃, and the like.

“Optionally substituted” means a substituent which is substituted independently with zero to three substituents selected from lower alkyl, halo, OH, cyano, amino, nitro, lower alkoxy, or halo-lower alkyl.

The definitions described herein may be appended to form chemically-relevant combinations, such as “heteroalkylaryl,” “haloalkylheteroaryl,” “arylalkylheterocyclyl,” “alkylcarbonyl,” “alkoxyalkyl,” and the like. When the term “alkyl” is used as a suffix following another term, as in “phenylalkyl,” or “hydroxyalkyl,” this is intended to refer to an alkyl group, as defined above, being substituted with one to two substituents selected from the other specifically-named group. Thus, for example, “phenylalkyl” refers to an alkyl group having one to two phenyl substituents, and thus includes benzyl, phenylethyl, and biphenyl. An “alkylaminoalkyl” is an alkyl group having one to two alkylamino substituents. “Hydroxyalkyl” includes 2-hydroxyethyl, 2-hydroxypropyl, 1-(hydroxymethyl)-2-methylpropyl, 2-hydroxybutyl, 2,3-dihydroxybutyl, 2-(hydroxymethyl), 3-hydroxypropyl, and so forth. Accordingly, as used herein, the term “hydroxyalkyl” is used to define a subset of heteroalkyl groups defined below. The term -(ar)alkyl refers to either an unsubstituted alkyl or an aralkyl group. The term (hetero)aryl or (het)aryl refers to either an aryl or a heteroaryl group.

Commonly used abbreviations include: acetyl (Ac), azo-bis-isobutyrylnitrile (AIBN), atmospheres (Atm), 9-borabicyclo[3.3.1]nonane (9-BBN or BBN), tert-butoxycarbonyl (Boc), di-tert-butyl pyrocarbonate or boc anhydride (BOC₂O), benzyl (Bn), butyl (Bu), Chemical Abstracts Registration Number (CASRN), benzyloxycarbonyl (CBZ or Z), carbonyl diimidazole (CDI), 1,4-diazabicyclo[2.2.2]octane (DABCO), diethylaminosulfur trifluoride (DAST), dibenzylideneacetone (dba), 1,5-diazabicyclo[4.3.0]non-5-ene (DBN), 1,8-diazabicyclo[5.4.0]undec-7-ene (DBU), N,N′-dicyclohexylcarbodiimide (DCC), 1,2-dichloroethane (DCE), dichloromethane (DCM), diethyl azodicarboxylate (DEAD), di-iso-propylazodicarboxylate (DIAD), di-iso-butylaluminumhydride (DIBAL or DIBAL-H), di-iso-propylethylamine (DIPEA), N,N-dimethyl acetamide (DMA), 4-N,N-dimethylaminopyridine (DMAP), N,N-dimethylformamide (DMF), dimethyl sulfoxide (DMSO), 1,1′-bis-(diphenylphosphino)ethane (dppe), 1,1-bis-(diphenylphosphino)ferrocene (dppf), 1-(3-dimethylaminopropyl)-3-ethylcarbodiimide hydrochloride (EDCI), ethyl (Et), ethyl acetate (EtOAc), ethanol (EtOH), 2-ethoxy-2H-quinoline-1-carboxylic acid ethyl ester (EEDQ), diethyl ether (Et₂O), O-(7-azabenzotriazole-1-yl)-N,N,N′N′-tetramethyluronium hexafluorophosphate acetic acid (HATU), acetic acid (HOAc), 1-N-hydroxybenzotriazole (HOBt), high pressure liquid chromatography (HPLC), iso-propanol (IPA), lithium hexamethyl disilazane (LiHMDS), methanol (MeOH), melting point (mp), MeSO₂— (mesyl or Ms), methyl (Me), acetonitrile (MeCN), m-chloroperbenzoic acid (MCPBA), mass spectrum (ms), methyl t-butyl ether (MTBE), N-bromosuccinimide (NBS), N-carboxyanhydride (NCA), N-chlorosuccinimide (NCS), N-methylmorpholine (NMM), N-methylpyrrolidone (NMP), pyridinium chlorochromate (PCC), pyridinium dichromate (PDC), phenyl (Ph), propyl (Pr), iso-propyl (i-Pr), pounds per square inch (psi), pyridine (pyr), room temperature (rt or RT), tert-butyldimethylsilyl or t-BuMe₂Si (TBDMS), triethylamine (TEA or Et₃N), 2,2,6,6-tetramethylpiperidine 1-oxyl (TEMPO), triflate or CF₃SO₂-(Tf), trifluoroacetic acid (TFA), 1,1′-bis-2,2,6,6-tetramethylheptane-2,6-dione (TMHD), O-benzotriazol-1-yl-N,N,N′,N′-tetramethyluronium tetrafluoroborate (TBTU), thin layer chromatography (TLC), tetrahydrofuran (THF), trimethylsilyl or Me₃Si (TMS), p-toluenesulfonic acid monohydrate (TsOH or pTsOH), 4-Me-C₆H₄SO₂— or tosyl (Ts), N-urethane-N-carboxyanhydride (UNCA). Conventional nomenclature including the prefixes normal (n), iso (i-), secondary (sec-), tertiary (tert-) and neo have their customary meaning when used with an alkyl moiety. (J. Rigaudy and D. P. Klesney, Nomenclature in Organic Chemistry, IUPAC 1979 Pergamon Press, Oxford.).

The metal ion(s) of the present disclosure are bound by the contrast agent. In some embodiments, the metal ion is Gadolinium (GdIII). In some embodiments, the metal ion is technetium. In some embodiments, the metal ion is indium. A person of skill in the art would understand that other metal ions suitable for use in contrast agents may also be used in the compounds of the present disclosure for example manganese, copper, copper 64 and iron.

The targeting portion of the contrast agent may be selected from the group of ethylenediaminetetraacetic acid (EDTA), 1,4,7,10-tetraazacyclododecane tetraacetic acid (DOTA), diethylene triaminopentaacetic acid DTPA, and/or Triglycollamic acid (NTA). In one embodiment, the targeting portion is DOTA. In another embodiment, the targeting portion is DTPA. A person of skill in the art would understand that other targeting portions including an aminocarboxylate functional group that are capable of binding necrotic tissue would also be suitable in preparation of contrast agents according to present disclosure.

The metal complexable portion of the contrast agent may be selected from the group of ethylenediaminetetraacetic acid (EDTA), 1,4,7,10-tetraazacyclododecane tetraacetic acid (DOTA), diethylene triaminopentaacetic acid DTPA, and/or Triglycollamic acid (NTA). Metal chelates are well known in the art and these compounds are often referred to as chelants, cheltors and chelating agents. The structure of the chelating agent is such that is forms a soluble, complex molecule with a metal ion and inactivates the metal ion from reacting with other elements or ions to produce precipitates. A person of skill in the art would understand that any chelating agent suitable for human administration would be suitable in the preparation of contrast agents according to present disclosure. In one embodiment, the metal complexable portion is DOTA. In another embodiment, the metal complexable portion is EDTA.

In one embodiment, the metal complexable portion is DOTA and the targeting portion DTPA. In an alternative embodiment, the both the metal complexable portion and the targeting portion are both DOTA.

The novel contrast agent compounds of the present disclosure may be prepared by any conventional means.

The novel contrast agents may exhibit some quantitative differences with respect to their properties in medical applications, such as blood clearance (ranging from relatively fast to relatively slow), elimination from the body (predominantly by kidney or shifted to hepatobiliary secretion), and plasma protein binding (from low to high). Labeling/complexation of the contrast agents may be accomplished, using methods well known in the art, by chelation with radioactive or non-radioactive metal ions, preferably with ions of an element with an atomic number selected from 21 to 32, 37 to 39, 42 to 44, 49, 50 or 57 to 83 such as for example: —Mn, Fe or Gd (with respect to non-radioactive metals), and −99 mTc, 111 in, 64Cu, 67Ga, 90Y, 188Re, 186Re and 163Dy (with respect to radioactive metals).

Chelation with metal ions may be performed by methods well documented in the literature, at any stage of the production of the novel class of contrast agents, although most often in the final step. When protected functional groups are present in the metal-complexable portion of the compound, they may be partly or completely deprotected prior to metal chelation. Ionizable groups not involved in metal complexation may be optionally neutralized by acidic or basic counter-ions or by (inorganic and/or organic) compounds bearing ionizable acidic and/or basic groups. Remaining acidic protons, for example those that have not been substituted by the metal ion, can optionally be completely or partially replaced by cations of inorganic or organic bases, basic amino-acids or amino-acid amides. Suitable inorganic counter ions are for example, the ammonium ion, the potassium ion, the calcium ion, the magnesium ion and, more preferably, the sodium ion. Suitable cations of organic bases are, among others, those of primary, secondary or tertiary amines, such as, for example, ethanolamine, diethanolamine, morpholine, glucamin, N,N-dimethylglucamine, tris(hydroxymethyl) aminomethane and especially N-methylglucamine. Suitable cations of amino-acids are, for example, those of lysine, arginine and ornithine as well as the amides of any other acidic or neutral amino-acid such as for example lysine methylamide, glycine ethylamide or serine methylamide.

Novel contrast agents according to present disclosure adhere to necrotic tissue, also referred to as dead tissue. On administration of the contrast agent, for example by intravenous injection, the contrast agent acts similar to a blood pool agent (also referred to as intravascular contrast agents). Following administration, a portion of the administered novel contrast agent binds to necrotic tissue and a portion of the administered contrast agent remains in plasma and is unbound. The portion of contrast agent in plasma is much greater that the portion bound to necrotic tissue. Thus, the novel contrast agents have a retention time, or half life, in plasma and a retention time, or half life, in necrotic tissue. The novel contrast agents demonstrate a similar retention time in plasma as compared to conventional contrast agents. For example, conventional contrast agents have a half life of about 30 to about 90 minutes, with virtually complete elimination of these agents within about 24 hours. Contrast agents of the present disclosure have a half life in plasma between about 30 minutes to 120 minutes, preferably 30 to 60 minutes. The contrast agent remaining in the plasma is eliminated via the urine. The portion of contrast agent bound to the necrotic tissue remains associated with the necrotic tissue for a period up to about 72 hrs. Traditional untargeted contrast agents are substantially cleared from a subject over a period of between 90 minutes and are almost completely eliminated after 24 hours. By comparison, the novel contrast agents of the present disclosure demonstrate a prolonged half life in tissue between about 48 to about 72 hours. The bound contrast agent highlights and improves visibility of necrotic tissue present.

In one embodiment, the long half life of novel contrast agent in necrotic tissue allows for the observation and identification of both the size and location of the necrotic tissue.

Tissue having suffered ischemic damage and cancerous tissue are not identifiable using MRI technology as these tissues appear similar to healthy tissue. The novel contrast agents of the present disclosure allow for both the observation and identification of the exact size and location of both infarcted tissue and cancerous tissue. In one aspect, the novel contrast agents facilitate the monitoring of death of cancerous tissue over time. In another aspect, the novel contrast agents facilitate improved patient care and enable a more precise medical diagnoses.

In some embodiments, contrast agents of the present disclosure may be used in vitro, in vivo and/or ex vivo, and may be administered directly or in the form of pharmaceutical compositions comprising the contrast agents in combination with at least one pharmaceutical acceptable carrier, as diagnostic agents and/or therapeutic agents. In one aspect, the contrast agents of the present disclosure are useful for the manufacture of compounds and/or medicaments suitable for use in diagnostic imaging or imaging-aided applications, including for example MRI, CT, SPECT, PET, MRI-aided applications, CT-aided applications, SPECT-aided applications or PET-aided applications. In another aspect, the contrast agents of the present disclosure are useful for the manufacture of diagnostic imaging agents or imaging-aided agents for use in the diagnostic imaging applications noted above. In a further aspect, the novel contrast agents may be used in vivo for visualizing and/or identifying organs, parts of organs, tissues, and parts of tissues for example necrotic tissue, and for visualizing and/or identifying diseases and pathologies. Contrast agents of the present disclosure may be useful in diagnosing diseases related to the presence of necrotic tissue. Such diseases that may be identified include ischemic insults for example myocardial or cerebral infarction, and space-occupying lesions for example tumors or inflammatory lesions that may be present in solid organs, for example the liver, kidney, spleen, and adrenal gland. Contrast agents of the present disclosure may be useful in differentiating between benign, pre-malignant or malignant tumors. These contrast agents may also be useful as a diagnostic tool in the evaluation of the effectiveness of a particular medical treatment, for instance in denoting the evolution or further evolution of necrosis.

In some embodiments, the contrast agents of the present disclosure may be useful in medical applications involving necrosis and necrosis-related pathologies, such as pathological or therapeutic necrosis caused by pathologic or therapeutically-induced ischemia or originating from trauma, radiation and/or chemicals, including therapeutic ablation, radiotherapy and/or chemotherapy, myocardial and cerebral infarctions. In this instance, the contrast agents are generally administered to a subject, intravenously, enterally or parenterally, as therapeutic and/or diagnostic agents. In one aspect, the novel contrast agent may be administered for use in the application of tumor ablation therapies, for example ischemic damage (i.e. pulmonary embolism, ischemic stroke, liver damage, kidney damage) to detect the extent of damage occurring in the affected tissue. The contrast agent binds to the necrotic tissue of the tumour and indicates to a medical practitioner the tumor size and location and in turn, allows for the continuous monitoring to track tumor size and indicate the effectiveness of a medical treatment method. The ability to monitor the effectiveness of an ongoing therapeutic treatment allows a subject to avoid undergoing ineffective medical treatment and in turn, helps to develop patient-specific therapy. This is of particular value in fields where a wide variety of potential therapeutics are available, for example in cancer treatment a wide number of chemotherapeutics are available. Continually monitoring tumor size through the use of the novel contrast agents allows for an earlier assessment of the effectiveness of a particular chemotherapy and in turn, allows a subject to avoid prolonged exposure to an ineffective line of treatment. The ability of the contrast agent to indicate the ineffectiveness of a medical treatment enables a medical practitioner to alter or change a course of medical treatment. Such a diagnostic tool allows for time saving measures and improvement of the overall patient outcome.

Pharmaceutically acceptable carriers for use in admixture with the contrast agents of the present disclosure are well known in the art and are selected based on the mode of administration of the contrast agent to the subject. In one aspect, a suitable formulation is a physiologically acceptable liquid formulation, preferably an aqueous solution or an emulsion or suspension including conventional surfactants such as polyethylene glycol.

In some embodiments, the contrast agents of the present disclosure provide a method for generating a diagnostic image of at least a part of a body of a subject following systemically or locally administering to the subject an effective amount of a contrast agent of the present invention. Preferably, the contrast agents of the present disclosure are used systemically as diagnostic agents by parenteral administration, including intravenous injection, at low doses. For example, when the metal ion of the contrast agent is gadolinium, a dosage range from about 10 to about 500 μmoles gadolinium per kg body weight, preferably from about 10 to about 200 μmoles gadolinium per kg body weight, more preferably from about 10 to about 100 μmoles gadolinium per kg body weight, and even more preferably from about 10 to about 50 μmoles gadolinium per kg body weight of the subject to be treated, wherein the gadolinium is bound to the metal-complexable portion of the contrast agent and the targeting portion is free to bind to necrotic tissue following administration of a contrast agent to a subject. In one aspect, the dose may comprise from about 5 μmoles/kg to about 1000 μmoles/kg (based on the mass of the subject), for example 5, 6, 7, 8, 9, 10, 15, 20, 25, 30, 35, 40, 45, 50, 55, 60, 65, 70, 75, 80, 85, 90, 95, 100, 120, 140, 160 180, 200, 250, 300, 350, 400, 450, 500, 750, 1000, μmoles/kg, or any amount therebetween; or from about 1 μmoles/kg to about 500 μmoles/kg or any amount therebetween, for example 1.0, 2.0, 5.0, 10.0, 15.0, 20.0, 25.0, 30.0, 35.0, 40.0, 45, 50.0, 55, 60.0, 65, 70.0, 75, 80.0, 85, 90.0, 95, 100, 120, 140, 160 180, 200, 250, 300, 350, 400, 450 500 μmoles/kg, or any amount therebetween; or from about 10 μmoles/kg to about 1000 ug/kg or any amount therebetween, for example 10.0, 11.0, 12.0 13.0, 14.0, 15.0, 20.0, 25.0, 30.0, 35.0, 40.0, 45, 50.0, 55, 60.0, 65, 70.0, 75, 80.0, 85, 90.0, 95, 100, 120, 140, 160 180, 200, 250, 300, 350, 400, 450, 500, 750, 1000 μmoles/kg, or any amount therebetween; or from about 20 μmoles/kg to about 1000 μmoles/kg or any amount therebetween, for example 20.0, 25.0, 30.0, 35.0, 40.0, 45, 50.0, 55, 60.0, 65, 70.0, 75, 80.0, 85, 90.0, 95, 100, 120, 140, 160 180, 200, 250, 300, 350, 400, 450, 500, 750, 1000 μmoles/kg.

Alternatively, the contrast agents of the present disclosure may also be useful for local administration, for example intracoronary administration in the case of a subject with myocardial infarction. Depending on the specific case, an effective local dose of the contrast agent of the present disclosure may be from about 0.1 to about 10 μmoles gadolinium per kg body weight, preferably from about 0.5 to about 7.5 μmoles gadolinium per kg body weight of the subject, more preferably from about 1 to about 5 μmoles gadolinium per kg body weight to be treated, wherein the gadolinium is bound to the metal-complexable portion of the contrast agent and the targeting portion is free to bind to necrotic tissue following administration of a contrast agent to a subject. In one aspect, the dose may comprise from about 0.1 μmoles/kg to about 10 μmoles/kg (based on the mass of the subject), for example 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 8.0, 8.5, 9.0, 9.5, 10.0 μmoles/kg, or any amount therebetween; or from about 0.5 μmoles/kg to about 7.5 μmoles/kg or any amount therebetween, for example 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5 μmoles/kg, or any amount therebetween; or from about 1 μmoles/kg to about 5 ug/kg or any amount therebetween, for example 0.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0 μmoles/kg, or any amount therebetween.

One of skill in the art will be readily able to interconvert the units as necessary, given the mass of the subject, the concentration of the pharmaceutical composition, individual components or combinations thereof, or volume of the pharmaceutical composition, individual components or combinations thereof, into a format suitable for the desired application.

The pharmaceutical compositions of the invention may include an “effective amount”, “therapeutically effective amount” or a “prophylactically effective amount” of a contrast agent of the invention. A “therapeutically effective amount” refers to an amount effective, at dosages and for periods of time necessary, to achieve the desired therapeutic result. A therapeutically effective amount of the contrast agent may be determined by a person skilled in the art and may vary according to factors such as the disease state, age, sex, and weight of the individual, and the ability of the contrast agent to elicit a desired response in the individual. A therapeutically effective amount is also one in which any toxic or detrimental effects of the antibody or antibody portion are outweighed by the therapeutically beneficial effects A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

In some embodiments, when a radioactive complexing metal such as indium-111 is used, the contrast agent may be administered with a radioactivity in the range of about 20 to 200 MBq (megabecquerels). When a radioactive complexing metal such as technetium-99 is used, the contrast agent may be administered with a radioactivity in the range of about 350 to 1,000 MBq.

Further aspects of the invention will become apparent from consideration of the ensuing description of the embodiments of the present disclosure. A person skilled in the art will realize that other embodiments of the invention are possible and that the details of the invention can be modified in a number of respects, all without departing from the inventive concept. Thus, the drawings, descriptions and examples are to be regarded as illustrative in nature and not restrictive.

### EXAMPLES

The compounds of the present disclosure can be prepared by any conventional means. Suitable processes for synthesizing these compounds are provided in the Examples below.

Reagents were purchased from Sigma Aldrich or other suppliers as indicated below however, reagents may also be purchased from other suppliers. Reactions were conducted using the equipment detailed below. The purification of the compounds was conducted by methods known to those skilled in the art, such as elution of silica gel column; however other methods may also be used. Compound identities were confirmed by mass spectrometry.

### Example 1

Compounds disclosed in WO 02/38546 were prepared following the methods outlined therein. Despite repeated efforts to synthesize a bis-gadolinium complex as disclosed the above-noted reference, the methods disclosed failed to produce a contrast agent demonstrating a stable, reproducible level of contrast in necrotic tissue. The compounds produced were subjected to extensive purification to exclude interference from impurities and/or isomers however, as the purity of the bis-gadolinium complex increased the corresponding level of contrast in necrotic tissue decreased. Further analysis and examination of the failed results led to the surprising discovery that the bis-gadolinium complex, which represented the major portion of the mixture of compounds produced, demonstrated minimal or no contrast in necrotic tissue. However, the mono-gadolinium complex, which represented a minor portion of the mixture of compounds produced and was considered an impurity, demonstrated high level of contrast in necrotic tissue. This discovery led to the further testing of the mono-gadolinium complexes and the development of the new class of novel contrast agents disclosed herein.

### Example 2

### Preparation of RF1002

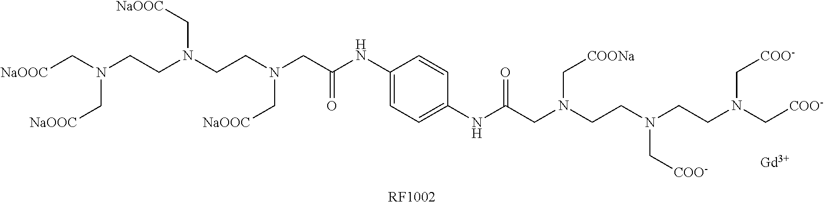

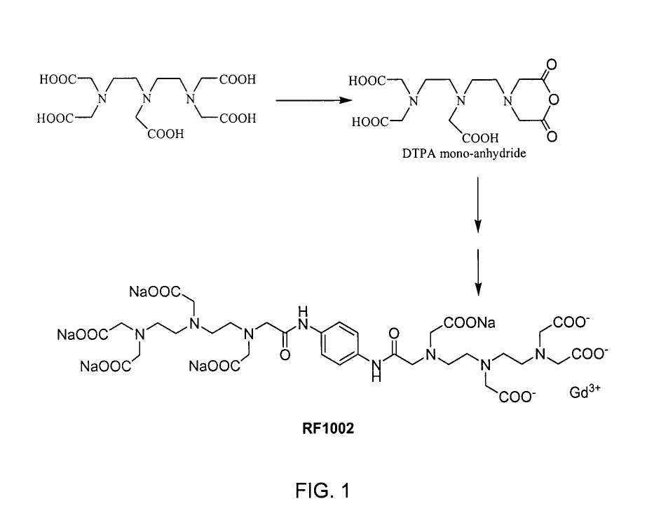

The RF 1002 compound was prepared following the process illustrated in FIG. 1.

DTPA mono-anhydride was synthesized with modification of the general procedure described by S. Halpern et al. (Labeling of monoclonal antibodies with indium 111 technique and advantages compared to radioiodine labeling. In “Radioimmunoimaging and Radioimmunotherapy,” ed. S. W. Burchiel and B. A. Rhodes, pp. 197-205 (1983). Elsevier Science Publ. Co. Inc., New York.)

DTPA (36 g) was added to 300 ml of trifluoroacetic acid. The mixture was heated for a period of 10 minutes to form a clear solution, and then cooled to room temperature. Thionyl chloride (12.3 g) was added. The mixture was then stirred and refluxed in with protection from moisture by means of a drying tube and the resulting reaction mixture heated using an oil bath and transformed from a yellow solid into solution that rapidly precipitated. The reflux of the mixture continued for one hour and then the volume was evaporated and reduced to remove the trifluoroacetic acid as much as possible. The residue was cooled and anhydrous ether (200 ml) was added. The solid was filtered and washed three times with 200-ml portions of anhydrous ether, and dried in an oven. Anhydride formation was confirmed by infrared spectroscopy which showed the presence of an anhydride carbonyl. The DTPA mono-anhydride residue was stored in a desiccated freezer as the anhydride is subject to hydrolysis.

The DTPA mono-anhydride (5.36 g, 14.3 mmol), K₂CO₃ (15.0 g) and DMSO (60 ml) were mixed in a single-necked flask and p-amino aniline (0.594 g, 5.5 mmol) was added at room temperature. The mixture was stirred for a period of 48 hours and then filtered. The volume of solution was reduced to one third under vacuum, and then 100 ml of toluene added. The resultant white solid residue was dried after filtration. The residue was purified by column chromatography using a preparative C 18 column (300 g). The column was eluted with distilled water. Purified fractions were combined and evaporated to dryness, yielding 1.2 g of RF 1002 ligand as white solid.

The ligand of RF1002 (1.0 mmol) was dissolved in water (60 ml) and Gadolinium (III) acetate (1.0 mmol) was added slowly. During the addition of Gadolinium (III) acetate, the pH was maintained at 7.4 with the addition of sodium hydroxide. Following the addition of Gadolinium (III) acetate, the mixture was stirred at room temperature over night, approximately 18 hours. The mixture was then applied to a C18-silicagel column that was rinsed with distilled water for desalting. Solvents were then removed in vacuo and the final RF1002 contrast agent product was obtained as a white solid. Identity of the product was confirmed by mass spectrometry.

### Example 3

### Preparation of RF1003

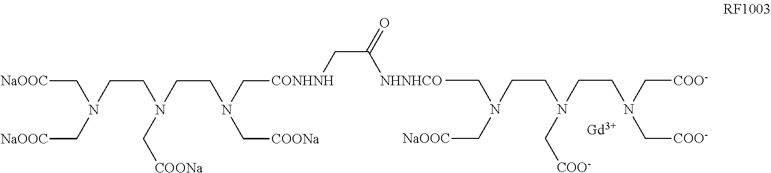

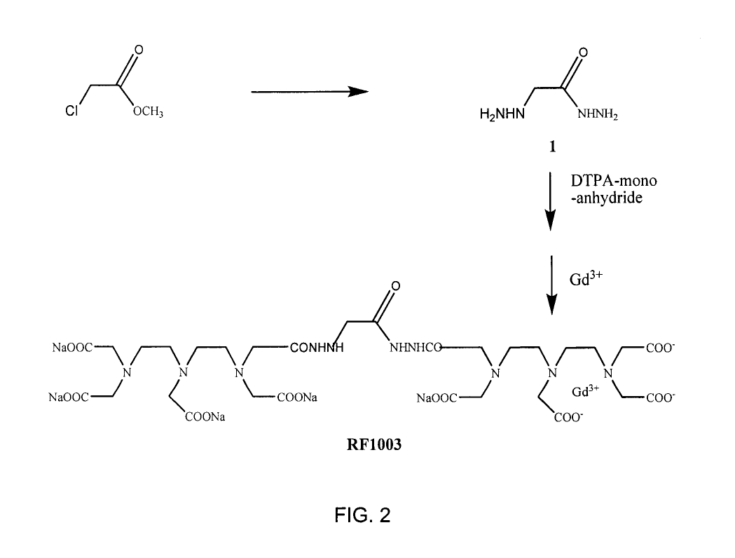

The RF 1003 compound was prepared following the process illustrated in FIG. 2.

Methyl chloroacetate (21.7 g, 20 mmol) and hydrazine monohydrate (100 g) were add to and dissolved in 70 ml of a mixture of pyridine (40 ml) and methanol (30 ml). The mixture was refluxed for a period of 48 hours. The solvents were then removed under a reduced pressure. The crude product was purified by crystallization in methanol-toluene and the resultant product, compound 1, was obtained in the form of colorless needles.

DTPA mono-anhydride (5.36 g, 14.3 mmol), K₂CO₃ (15 g) and DMSO (60 ml) were mixed in a single-necked flask and compound 1 (0.57 g, 5.5 mmol) was slowly added at room temperature. The mixture was stirred for a period of 24 hours and then filtered. The volume of solution was reduced to one third by rotary evaporation under vacuum, and then 100 ml of toluene was added. The resultant white solid residue was dried after filtration. The residue was purified by column chromatography using a preparative C 18 column (300 g). The column was eluted with distilled water. Purified fractions were combined and evaporated to dryness, yielding 0.8 g of RF1003 ligand as white solid.

The ligand of RF1003 (1.0 mmol) was dissolved in water (60 ml) and Gadolinium (III) acetate (1.0 mmol) was added slowly. During the addition of Gadolinium (III) acetate, the pH was maintained at 7.4 with the addition of sodium hydroxide. Following the addition of Gadolinium (III) acetate, the mixture was stirred at room temperature overnight, approximately 18 hours. The mixture was then applied on a C18-silicagel column that was eluted with distilled water for desalting. Solvents were removed in vacuo and final RF1003 contrast agent product was obtained as a white solid. Identity of the product was confirmed by mass spectrometry.

### Example 4

### Preparation of RF 1004

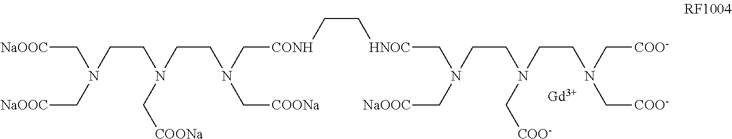

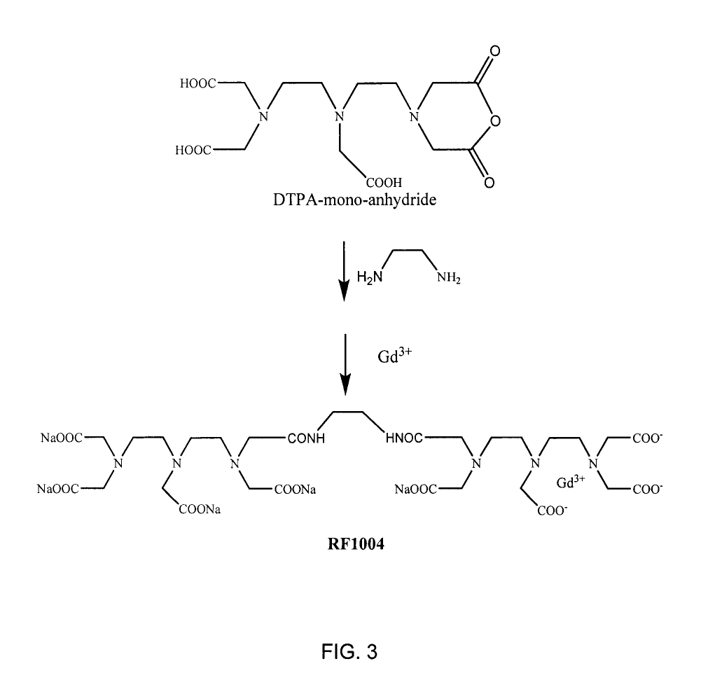

The RF 1004 compound was prepared following the process illustrated in FIG. 3.

DTPA mono-anhydride was synthesized with modification of the general procedure described by S. Halpern et al. (1983) as outlined in Example 1.

DTPA mono-anhydride (5.36 g, 14.3 mmol), K₂CO₃ (15 g) and DMSO (60 ml) were mixed in a single-necked flask and ethylenediamine (0.33 g, 5.5 mmol) was slowly added at room temperature. The mixture was stirred for a period of 24 hours and then filtered. The solution was reduced to one third by rotary evaporation under vacuum, and then 100 ml of toluene was added. The resultant white solid residue was dried after filtration. The residue was purified by column chromatography using a preparative C 18 column (300 g). The column was eluted with distilled water. Purified fractions were combined and evaporated to dryness, yielding 0.9 g of RF 1004 ligand product as a light yellow solid.

The ligand of RF1004 (1.0 mmol) was dissolved in water (60 ml) and Gadolinium (III) acetate (1.0 mmol) was added slowly. During the addition of Gadolinium (III) acetate, the pH was maintained at 7.4 with the addition of sodium hydroxide. Following the addition of Gadolinium (III) acetate, the mixture was stirred at room temperature overnight, approximately 18 hours. The mixture was then applied on a C18-silicagel column that was eluted with distilled water for desalting. Solvents were removed in vacuo and the final RF1004 contrast agent product was obtained as a white solid. Identity of the product was confirmed by mass spectrometry.

### Example 5

### Preparation of RF 1005

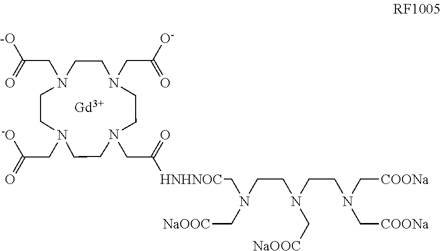

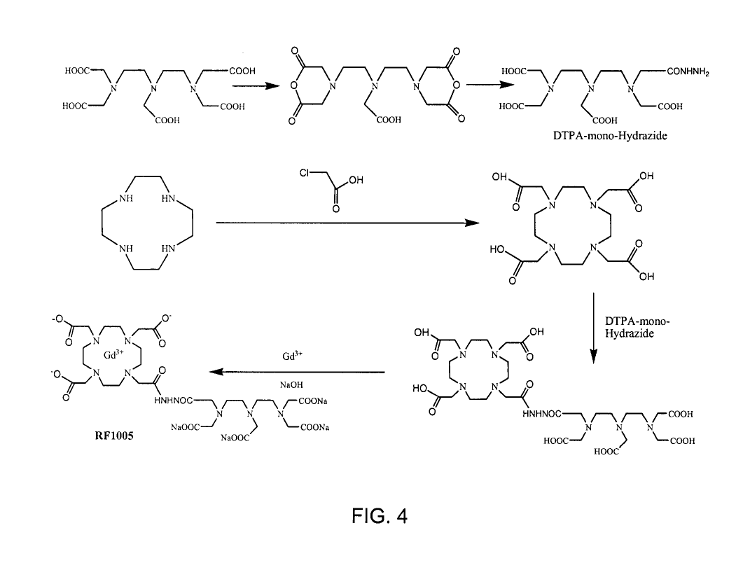

The RF 1005 compound was prepared following the process illustrated in FIG. 4.

Diethylenetriaminepentaacetic acid (39.3 g, 0.1 mole) was suspended in pyridine (50 g), and then acetic anhydride (40.8 g. 0.4 mole) was added. The mixture was then heated to 65° C. and the temperature was maintained for a period of 24 hours. The product DTPA-di-anhydride, was filtered, washed with acetic anhydride and ether, and dried.

DTPA mono-hydrazide was synthesized following the general procedure described by Clive Jolley et al (AppL Radiat. Isot. Vol. 47, No. 7, pp. 623-626, 1996). Cyclic diethylenetriaminepentaacetic acid anhydride (10 g) was added to water (100 ml) and hydrazine (2.2 ml) was added. The solution was stirred at room temperature for a period of 5 hours, and then evaporated to dryness under reduced pressure. The glassy residue was then triturated with diethyl ether (200 ml) to form a white powder, which was collected by filtration and dried under vacuum. The solid was then dissolved in water (20 ml) to form a solution and was purified by anion exchange chromatography. For anion exchange, the solution (20 ml) was loaded onto a column of Dowex-1 1×8-200 resin (500 ml bed volume) that had been washed with 1 M formic acid followed by water. Three fractions were eluted; fraction 1 with water (2.5 L), fraction 2 with 0.2 M formic acid (2.5 L) and fraction 3 with 1 M hydrochloric acid (2.5 L). Fraction 2 was then evaporated to dryness to yield the product, DTPA mono-hydrazide, as a white powder in 55% yield.

1,4,7,10-Tetraazacyclododecane Hydrochloride Salt (cyclen HCl salt, 4.0 g, 12.7 mmol) was dissolved in distilled water (20 ml). The pH was adjusted to 8.5 by the addition of 6N potassium hydroxide (KOH). Chloroacetic acid (5.4 g, 57 mmol) was added and the mixture was then heated to a temperature of 75° C. The temperature was maintained for a period of 24 h while its pH was concurrently maintained at a pH between 9 to 10 by the addition of 6N KOH. The mixture was then cooled to room temperature, and 6N HCl was added in an amount to adjust the pH of the mixture to 2. The temperature of the suspension was then cooled to 4° C. and the temperature was maintained for a period of 4 hours and then filtered. The crude product was re-crystallised in 6N HCl and the resultant product, 2,2′,2″,2′″-(1,4,7,10-tetraazacyclododecane-1,4,7,10-tetrayl)tetraacetic acid (DOTA) (4.5 g, 88% yield) was obtained as white solid (m.p. 264° C.).

DOTA (2.2 g, 5 mmol) was dissolved in distilled water (100 ml). NaOH was added in a quantity sufficient to adjust the pH of the mixture to 4.8. The solution was cooled to a temperature of 4° C. and stirred. N-Ethyl-N′-(3-dimethylaminopropyl) carbodiimide hydrochloride (EDCI, 1.92 g 10 mmol) was then added to the mixture followed by DTPA-mono-hydrazide (2.85 g, 7 mmol). The mixture was then stirred at 4° C. for a period of 1 hour. The temperature was then raised to room temperature, and was maintained for a period of 24 hours. Purification was conducted by the application of a preparative C 18 column (300 g). The column was eluted with distilled water. Pure fractions were combined and evaporated to dryness, yielding 0.5 g of RF1005 ligand as a white solid.

The ligand of RF1005 (1.0 mmol) was dissolved in water (60 ml) and Gadolinium (III) acetate (1.0 mmol) was added slowly. During the addition of Gadolinium (III) acetate, the pH was maintained at 7.4 with the addition of sodium hydroxide. After addition of Gadolinium (III) acetate, the mixture was refluxed over night, approximately 18 hours. The mixture was then applied on a C18-silicagel column that was eluted with distilled water for desalting. Solvents were removed in vacuo and the final RF1005 contrast agent product was obtained as a brown solid. Identity of the product was confirmed by mass spectrometry.

### Example 6

### Preparation of RF 1006

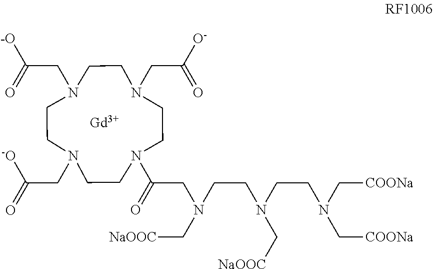

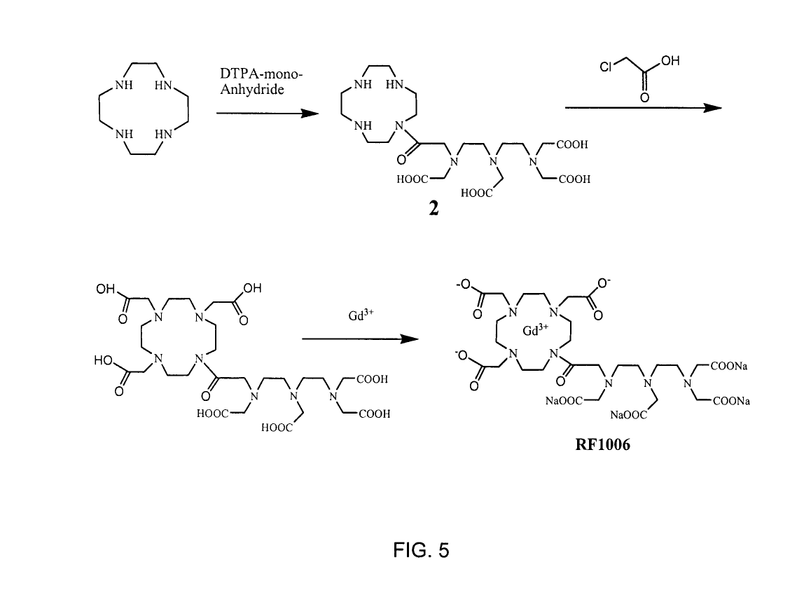

The RF 1006 compound was prepared following the process illustrated in FIG. 5.

1,4,7,10-Tetraazacyclododecane Hydrochloride Salt (cyclen, 1.9 g, 6 mmol) was dissolved in distilled water (60 ml) and the pH was adjusted to 8.5 by addition of 6N sodium hydroxide (NaOH). The solution was cooled to a temperature of 4° C. and DOPA-mono-anhydride (3.0 g, 8 mmol) was added. The mixture was stirred for a period of 2 hours at a temperature of 4° C. The temperature was then raised to room temperature, and maintained for a period of 48 hours. The mixture was purified by column chromatography using a preparative C 18 column (300 g). The column was eluted with distilled water. Purified fractions were combined and evaporated to dryness to yield 2.4 g of compound 2 in the form of yellow solid.

Compound 2 (2.0 g, 3.7 mmol) was dissolved in distilled water (60 ml) and the pH was adjusted to 8 by addition of KOH. Chloroacetic acid (2.1 g, 22.3 mmol) was then added, and the mixture was heated to 70° C. The temperature was maintained for a period of 24 h while its pH was concurrently maintained between 8 to 9 by the addition of 6N KOH. The mixture was then cooled and 6N HCl was added to adjust the mixture to pH 2. The temperature was then adjusted to 4° C. and the temperature was maintained for a period of 4 hours, and was then filtered. The mixture was purified by column chromatography using a preparative C 18 column (300 g). The column was eluted with distilled water. Purified fractions were combined and evaporated to dryness, yielding 2.4 g of RF1006 ligand as a brown solid.

The ligand of RF1006 (1.0 mmol) was dissolved in water (60 ml) and Gadolinium (III) acetate (1.0 mmol) was added slowly. During the addition of Gadolinium (III) acetate, the pH was maintained at 7.4 with the addition of sodium hydroxide. After addition Gadolinium (III) acetate, the mixture was refluxed over nigh, approximately 18 hours. The mixture was then applied on a C18-silicagel column that was rinsed with distilled water for desalting. Solvents were removed in vacuo and the final RF 1006 contrast agent product was obtained as a brown solid. Identity of the product was confirmed by mass spectrometry.

### Example 7

### Preparation of RF 1101

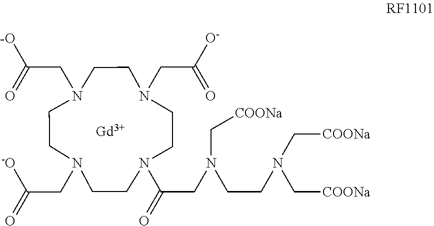

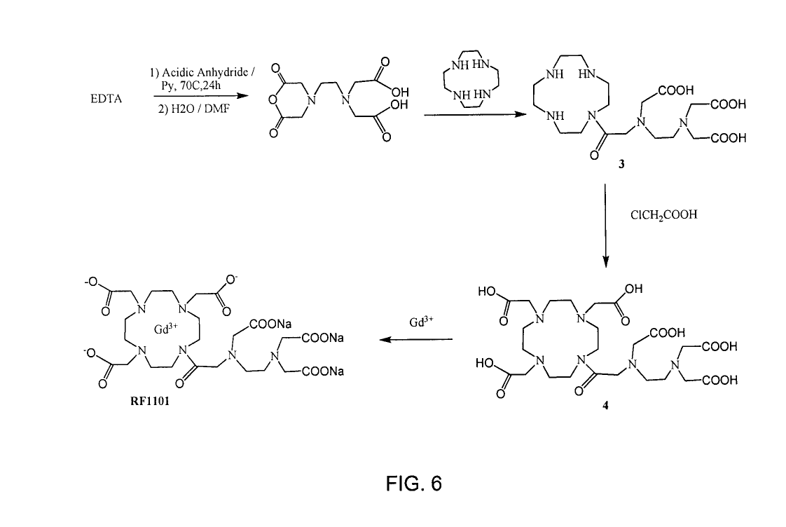

The RF 1101 compound was prepared following the process illustrated in FIG. 6.

EDTA mono-anhydride was prepared according to the literature method outlined in the Helvetica Chimica Acta. (1997, 80, 1183-1189). Cyclen (1.88 g, 10 mmol) was dissolved in anhydrous chloroform (100 ml) at a temperature of 60° C., and then EDTA mono-anhydride (4.28 g, 12 mmol) was added slowly. The reaction was monitored until all the cyclen was consumed. The solvent was then removed by distillation and the resultant crude EDTA mono-anhydride product, compound 3, was not purified.

Compound 3 (4.0 mmol) was dissolved in distilled water (60 ml) and KOH was added to adjust the pH to 8. Chloroacetic acid (24.0 mmol) was then added. The mixture was then heated to a temperature of 75° C. The temperature was maintained for a period of 24 h while the pH was concurrently maintained between 8 to 9 by the addition of 6N KOH. The mixture was then cooled and 6N HCl was added to adjust the mixture to pH 2. The temperature was then adjusted to 4° C. and the temperature was maintained for a period of 4 hours, and was then filtered. The mixture was purified by column chromatography using a preparative C 18 column (300 g). The column was eluted with distilled water. Purified fractions were combined and evaporated to dryness yielding compound 4, RF1101 ligand 1.5 g.

The ligand of RF1101 (1.0 mmol) was dissolved in water (60 ml) and Gadolinium (III) acetate (1.0 mmol) was added slowly. During the addition of Gadolinium (III) acetate, the pH was maintained at 7.4 with the addition of sodium hydroxide. After addition Gadolinium (III) acetate, the mixture was refluxed over night, approximately 18 hours. The mixture was applied to a C18-silicagel column that was eluted with distilled water for desalting. Solvents were removed in vacuo and the final RF1101 contrast agent product was obtained as a brown solid. Identity of the product was confirmed by mass spectrometry.

### Example 8

### Preparation of RF 1102

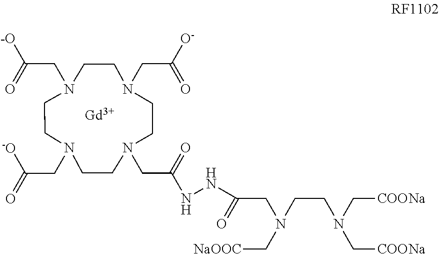

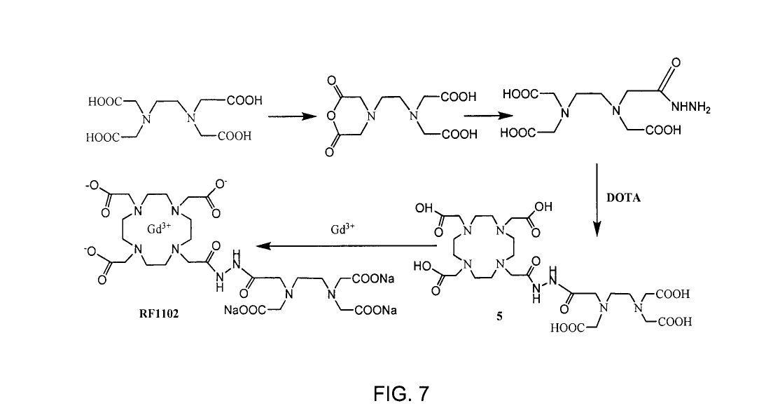

The RF 1102 compound was prepared following the process illustrated in FIG. 7.

EDTA monohydrazide was synthesized following the general procedure described by Clive Jolley et al. (Appl. Radiat. Isot. Vol. 47, No. 7, pp. 623-626, 1996). EDTA monoanhydride (10.0 g) was dissolved in water (100 ml) and then hydrazine (3.2 ml) was added. The solution was stirred at room temperature over night, approximately 18 hours, and then evaporated to dryness under reduced pressure. The glassy residue was triturated with diethyl ether to form a white powder, which was collected by filtration and dried under vacuum. The solid residue was dissolved in water (20 ml) to form a solution and was purified by anion exchange chromatography. For the anion exchange, the solution (20 ml) was loaded onto a column of Dowex-1 1×8-200 resin (500 ml bed volume) that was washed with 1 M formic acid followed by water. Three fractions were eluted, fraction 1 with water (2.5 L), fraction 2 with 0.2 M formic acid (2.5 L) and fraction 3 with 1 M hydrochloric acid (2.5 L). Fraction 2 was then evaporated to dryness to yield the EDTA-mono-hydrazide product as a white powder in 50 to 60% yield.

DOTA (2.2 g, 5 mmol) was dissolved in of distilled water (100 ml) and NaOH was added to adjust the pH to 4.8. The mixture was then cooled to a temperature of 4° C. and stirred for a period of 30 minutes. N-Ethyl-N′-(3-dimethylaminopropyl) carbodiimide hydrochloride (EDCI, 1.92 g 10 mmol) was then added to the mixture followed by EDTA-mono-hydrazide (7 mmol). The mixture was then stirred for a period of 1 hour and the temperature was maintained at 4° C. The temperature was then adjusted to room temperature for a period of 24 hours. The mixture was purified by column chromatography using a preparative C 18 column (300 g). The column was eluted with distilled water. Purified fractions were combined and evaporated to dryness, yielding 0.5 g of RF1102 ligand, compound 5, as a white solid.

The ligand of RF1102 (1.0 mmol) was dissolved in water (60 ml) and Gadolinium (III) acetate (1.0 mmol) was added slowly. During the addition of Gadolinium (III) acetate, the pH was maintained at 7.4 with the addition of sodium hydroxide. After addition of Gadolinium (III) acetate, the mixture was refluxed over night, approximately 18 hours. The mixture was applied to a C18-silicagel column that was eluted with distilled water for desalting. Solvents were removed in vacuo and the final RF1102 contrast agent product was obtained as a brown solid. Identity of the product was confirmed by mass spectrometry.

### Example 9

### Preparation of RF 1103

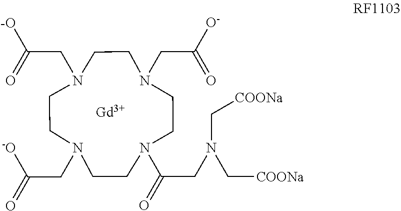

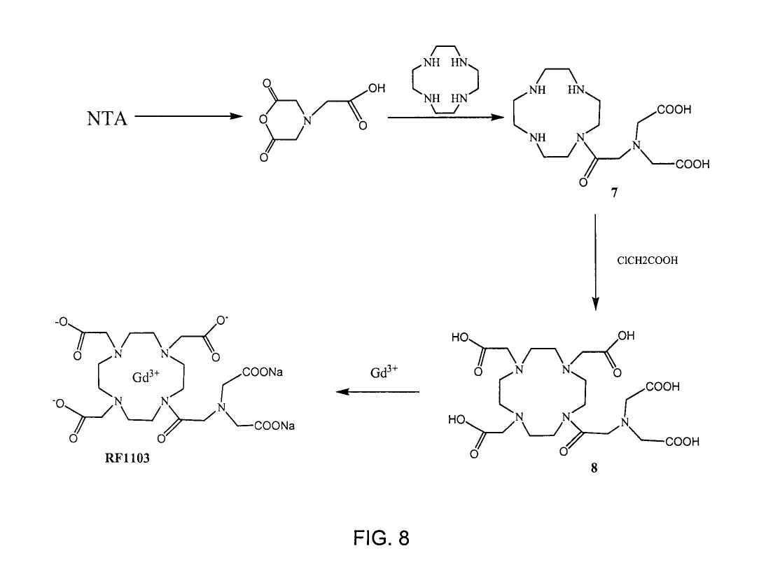

The RF 1103 compound was prepared following the process illustrated in FIG. 8.

NTA anhydride was synthesized following the general procedure outlined in U.S. Pat. No. 3,621,018 (Raymong R. Hindersinn et al.). Cyclen (1.88 g, 10 mmol) was dissolved in anhydrous chloroform (100 ml) at temperature of 60° C. NTA anhydride (12 mmol) was then added and the reaction mixture was stirred and the temperature was maintained until all cyclen was consumed. The solvent was then removed by distillation and the resultant crude NTA anhydride product, compound 7, was not purified.

Compound 7 (4.0 mmol) was dissolved in distilled water (60 ml) and KOH was added to adjust the pH to 8. Chloroacetic acid (24.0 mmol) was then added and the mixture was heated to a temperature of 75° C.

The temperature was maintained for a period of 24 h while the pH was concurrently maintained between 8 to 9 by the addition of 6N KOH. The mixture was then cooled to room temperature and 6N HCl was added to adjust the mixture to pH 2. The temperature was then adjusted to 4° C. and the temperature was maintained for a period of 4 hours. The mixture was then filtered. The mixture was purified by column chromatography using a preparative C 18 column (300 g). The column was eluted with distilled water. Purified fractions were combined and evaporated to dryness yielding 1.3 g of RF1003 ligand, compound 8.

The ligand of RF1103 (1.0 mmol) was dissolved in water (60 ml) and Gadolinium (III) acetate (1.0 mmol) was added slowly. During the addition of Gadolinium (III) acetate, the pH was maintained at 7.4 with the addition of sodium hydroxide. After addition of Gadolinium (III) acetate, the mixture was refluxed over night, approximately 18 hours. The mixture was applied to a C18-silicagel column that was rinsed with distilled water for desalting. Solvents were removed in vacuo and the final RF 1103 contrast agent product was obtained as a brown solid. Identity of the product was confirmed by mass spectrometry.

### Example 10

### Preparation of RF 1104

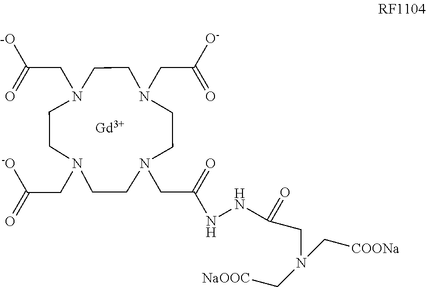

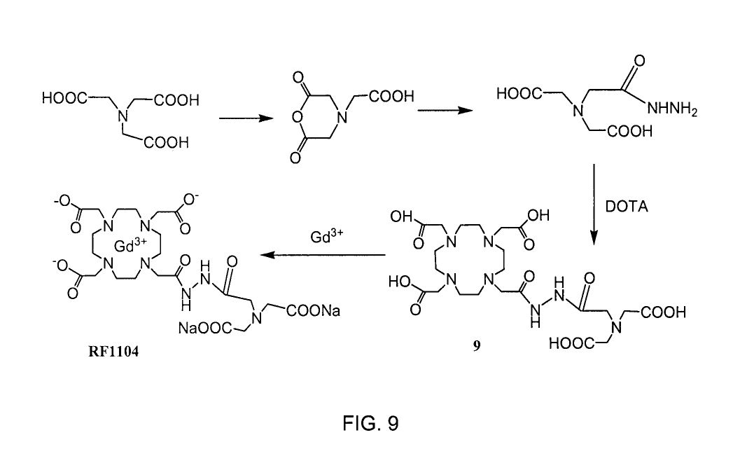

The RF 1104 compound was prepared following the process illustrated in FIG. 9.

NTA mono-anhydride (10 g) was added to water (100 ml) followed by the addition of hydrazine (3.2 ml). The solution was stirred at room temperature over night, approximately 18 hours and then evaporated to dryness under reduced pressure. The glassy residue was then triturated with diethyl ether to form a white powder, which was collected by filtration and dried under vacuum. The residue was then dissolved in water (20 ml) to form a solution and was purified by anion exchange chromatography. The solution (20 ml) was loaded onto a column of Dowex-1 1×8-200 resin (500 ml bed volume) that was washed with 1 M formic acid followed by water. Three fractions were eluted, fraction 1 with water (2.5 L), fraction 2 with 0.2 M formic acid (2.5 L) and fraction 3 with 1 M hydrochloric acid (2.5 L). Fraction 2 was then evaporated to dryness to yield the product, NTA-mono-hydrazide, as a white powder in a 55% yield.

DOTA (2.2 g, 5 mmol) was dissolved in distilled water (100 ml). NaOH was then added to the mixture to adjust the pH to 4.8. The solution was then cooled to a temperature of 4° C. and stirred for a period of 30 minutes. N-Ethyl-N′-(3-dimethylaminopropyl) carbodiimide hydrochloride (EDCI, 1.92 g 10 mmol) was then added to the mixture followed by NTA-mono-hydrazide (7 mmol). The mixture was stirred for a period of 1 hour and the temperature was maintained at 4° C. The temperature was then adjusted to room temperature and maintained for a period of 24 hours. The mixture was purified by column chromatography using a preparative C 18 column (300 g). The column was eluted with distilled water. Purified fractions were combined and evaporated to dryness, yielding the RF1104 ligand, compound 9, as a white solid.

The ligand of RF1104 (1.0 mmol) was dissolved in water (60 ml) and Gadolinium (III) acetate (1.0 mmol) was added slowly. During the addition of Gadolinium (III) acetate, the pH was maintained at 7.4 with the addition of sodium hydroxide. After addition of Gadolinium (III) acetate, the mixture was refluxed over night, approximately 18 hours. The mixture was applied on a C18-silicagel column that was rinsed with distilled water for desalting. Solvents were removed in vacuo and the final RF 1004 contrast agent product was obtained as a brown solid. Identity of the product was confirmed by mass spectrometry.

### Example 11

### Preparation of RF 1105

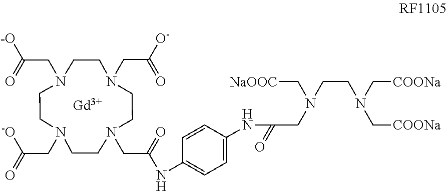

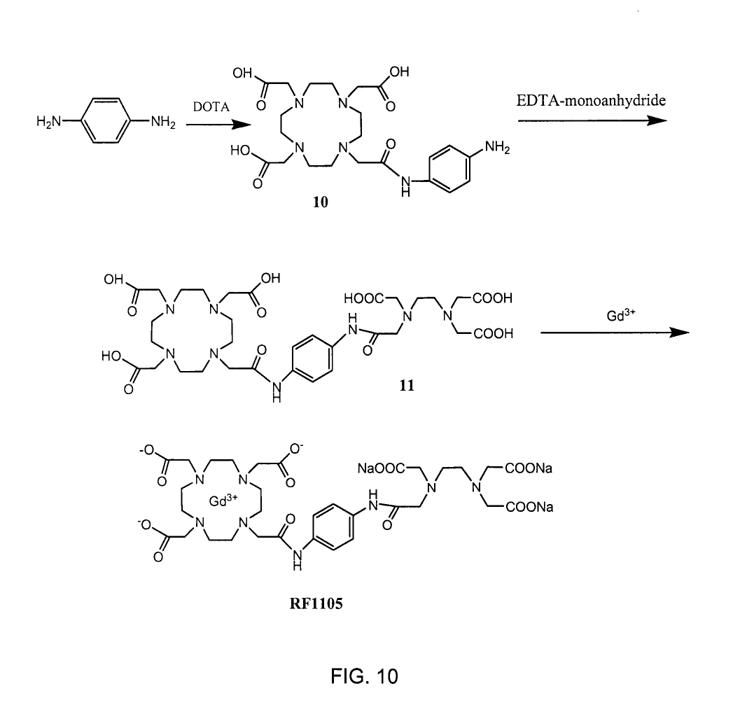

The RF 1105 compound was prepared following the process illustrated in FIG. 10.

DOTA (2.2 g, 5 mmol) was dissolved in of distilled water (100 ml). NaOH was then added to adjust the pH to 4.8. The solution was then cooled to a temperature of 4° C. and stirred for a period of 30 minutes. N-Ethyl-N′-(3-dimethylaminopropyl) carbodiimide hydrochloride (EDCI, 1.92 g 10 mmol) was then added to the mixture followed by p-amino aniline (7 mmol). The mixture was then stirred for a period of 1 hour and then filtered. The temperature was then adjusted to room temperature and maintained for a period of 24 hours. The mixture was then purified by column chromatography using a preparative C 18 column (300 g). The column was eluted with 10% methanol in water. Purified fractions were combined and evaporated to dryness, yielding the product, compound 10 as a white solid.

EDTA mono-anhydride (15.0 mmol), K₂CO₃ (15.0 g) and DMSO (60 ml) were mixed in a single-necked flask and compound 10 (5.0 mmol) was slowly added at room temperature. The mixture was then stirred for a period of 48 hours and then filtered. The volume of solution was then reduced to one third by rotary evaporation under vacuum, and then 100 ml of toluene was added. The resultant white solid residue was then dried following filtration. The mixture was purified by column chromatography using a preparative C 18 column (300 g). The column was eluted with distilled water. Purified fractions were combined and evaporated to dryness, yielding the RF1105 ligand, compound 11 as white solid.

The ligand of RF1105 (1.0 mmol) was dissolved in water (60 ml) and Gadolinium (III) acetate (1.0 mmol) was added slowly. During the addition Gadolinium (III) acetate, the pH was maintained at 7.4 with the addition of sodium hydroxide. After addition of Gadolinium (III) acetate the mixture was refluxed over night, approximately 18 hours. The mixture was applied on a C18-silicagel column that was rinsed with distilled water for desalting. Solvents were removed in vacuo and the RF1105 contrast agent product was obtained as a white solid. Identity of the product was confirmed by mass spectrometry.

### Example 12

### Preparation of RF 1107

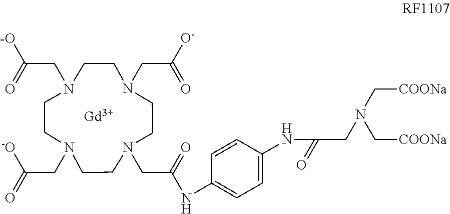

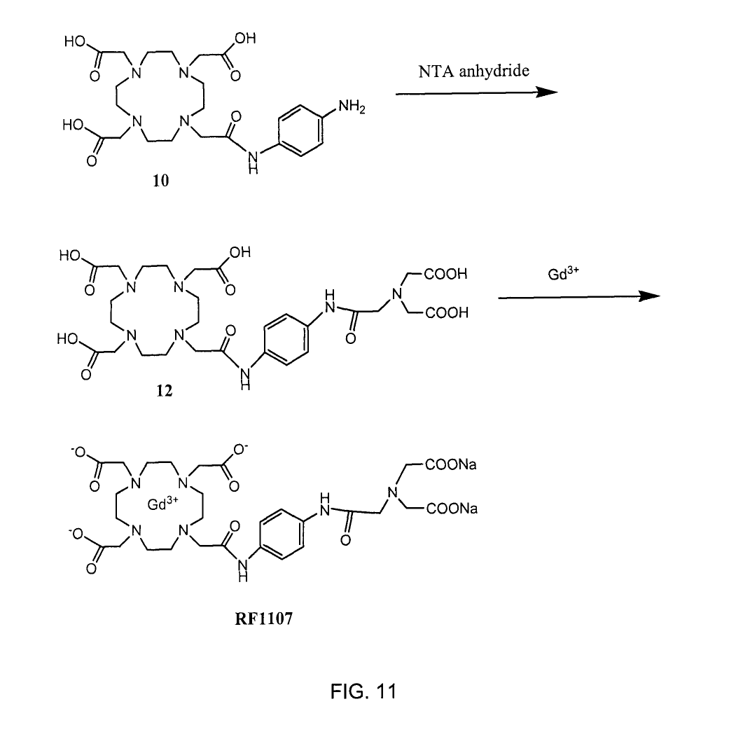

The RF 1107 compound was prepared following the process illustrated in FIG. 11.

NTA anhydride (15.0 mmol), K₂CO₃ (15.0 g) and DMSO (60 ml) were mixed in a single-necked flask and compound 10 (5.0 mmol) was slowly added at room temperature. The mixture was then stirred for a period of 48 hours and then filtered. The volume of solution was reduced to one third by rotary evaporation under vacuum, and then 100 ml of toluene was added. The resultant white solid residue was then dried following filtration. The mixture was purified by column chromatography using a preparative C 18 column (300 g). The column was eluted with distilled water. Purified fractions were combined and evaporated to dryness, yielding RF1107 ligand, compound 12 as a white solid.

The ligand of RF1107 (1.0 mmol) was dissolved in water (60 ml) and Gadolinium (III) acetate (1.0 mmol) was added slowly. During the addition of Gadolinium (III) acetate, of the pH was maintained at 7.4 with sodium hydroxide. After addition of Gadolinium (III) acetate the mixture was refluxed over night, approximately 18 hours. The mixture was applied on a C18-silicagel column that was rinsed with distilled water for desalting. Solvents were removed in vacuo and the RF 1107 contrast agent product was obtained as a white solid. Identity of the product was confirmed by mass spectrometry.

### Example 13

### Preparation of RF 1201

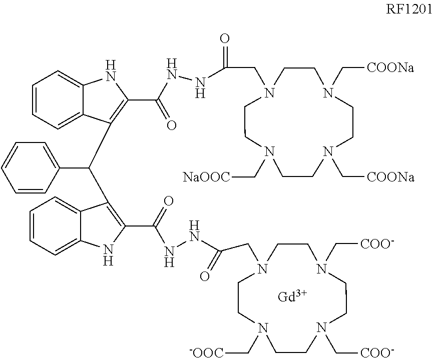

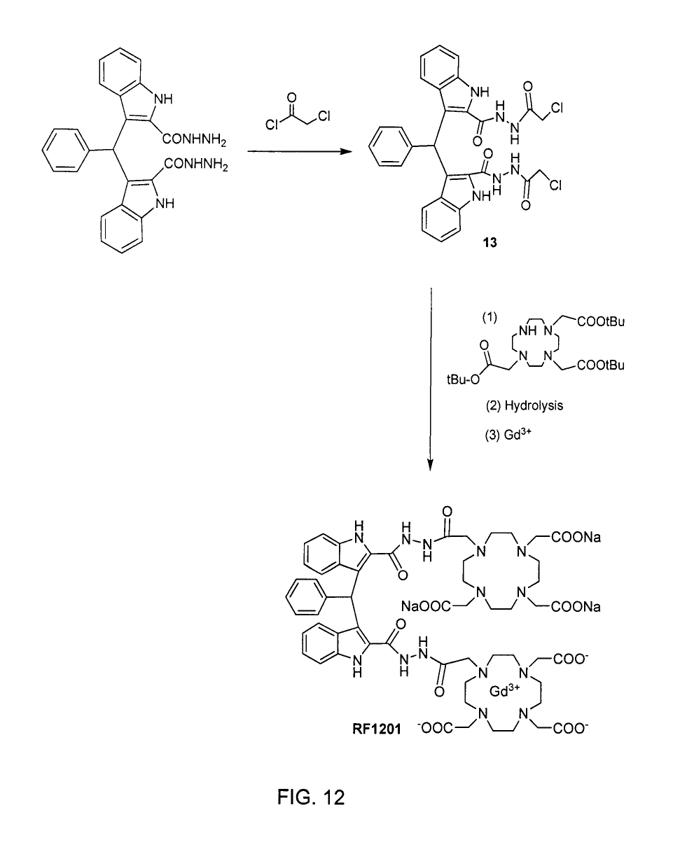

The RF 1201 compound was prepared following the process illustrated in FIG. 12.

Compound 13:

Compound 13 was synthesized by the dropwise addition of chloroacetyl chloride to an ice cold solution of bis-indole-hydrazide in DMF and then stirred at room temperature for one hour. The contents in the reaction flask were then poured slowly into sodium bicarbonate solution and the resulting precipitates were filtered, washed and dried in an oven at 110° C. The dried product (Yield: 80%) was obtained as a white solid. m/z (FAB) 589, 591, 593 (M-H).

RF 1201 Ligand:

To DO3A triester hydrobromide (2.8 g, 5 mmol) in acetonitrile (40 ml) stirred under nitrogen, was added triethylamine (0.50 g, 5 mmol) and compound 13 (1.18 g, 2 mmol). The mixture was heated to a gentle reflux for 48 h. The reaction mixture was then cooled, filtered and the filtrate evaporated to dryness under reduced pressure to leave a yellow gum. The product was dissolved in a minimum volume of chloroform and then chromatographed through silica gel, using 20:1:1 chloroform:methanol: isopropylamine as eluant, to yield a yellow solid. The crude was dissolved in dichloromethane (20 ml) and to the solution was carefully added trifluoroacetic acid (20 ml). The solution was left stirring at room temperature for 24 h and the solvents then removed under reduced pressure. Dichloromethane (40 ml) was then added and evaporated off twice, followed by two similar treatments with ether. The solid residue was taken up in a minimum of DMF and THF added dropwise, until the solution just turned cloudy, then, left to stand overnight. The crystalline powder was collected, washed with THF (2×20 ml) and dried to afford a pale yellow powder.

RF 1201:

the RF1201 ligand (1.0 mmol) was dissolved in water (60 ml) and Gadolium (III) acetate (1.0 mmol) was added slowly. During the addition the pH was maintained at 7.4 with sodium hydroxide. After addition the mixture was refluxed overnight. For desalting the mixture was applied on a C18-silicagel column that was rinsed with distilled water. Solvents were removed in vacuo and product was obtained as a white solid. Identity of the product was confirmed by mass spectrometry.

### Example 14

### Preparation of RF 1202

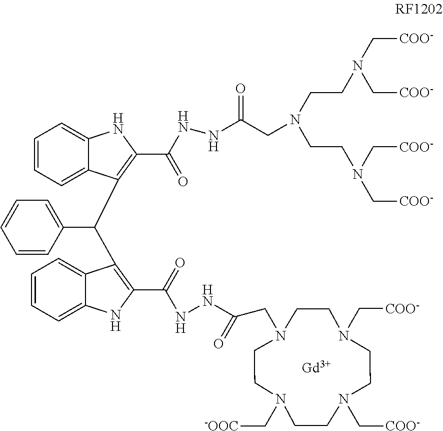

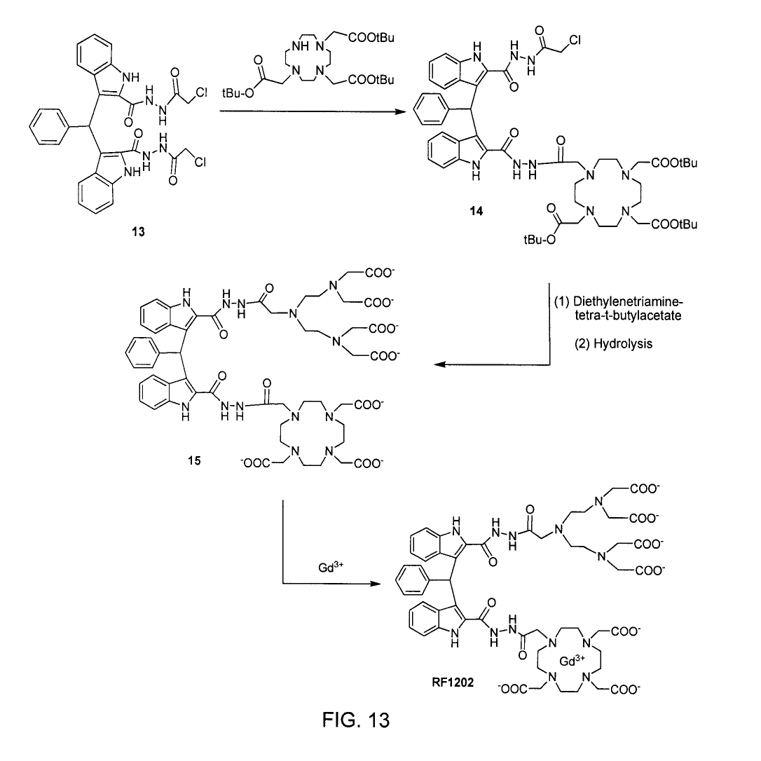

The RF 1202 compound was prepared following the process illustrated in FIG. 13.

Compound 14:

To a mixture of compound 13 (1.18 g, 2 mmol) and triethylamine (0.20 g, 2 mmol)) in acetonitrile (40 ml) stirred under nitrogen, was added DO3A triester hydrobromide (1.12 g, 2 mmol) (as discussed in Bryson, J. M., Bioconjugate Chemistry, 2008, 19(8), 1505-1509). The mixture was heated to a gentle reflux for 48 h. The reaction mixture was then cooled, filtered and the filtrate evaporated to dryness under reduced pressure to leave a yellow gum. The product was dissolved in a minimum volume of chloroform and then chromatographed through silica gel, using 20:1:1 chloroform:methanol: isopropylamine as eluant, to yield a yellow solid.

RF 1202 Ligand (Compound 15):

To a mixture of compound 14 (2.13 g, 2 mmol) and triethylamine (0.20 g, 2 mmol)) in acetonitrile (40 ml) stirred under nitrogen, was added diethylenetriamine-tetra-t-butylacetate (1.23 g, 2.2 mmol). The mixture was heated to a gentle reflux for 48 h. The reaction mixture was then cooled, filtered and the filtrate evaporated to dryness under reduced pressure to leave a yellow gum. The product was dissolved in a minimum volume of chloroform and then chromatographed through silica gel, using 3% methanol-dichloromethane as eluant, to yield a yellow solid. This solid was taken up in THF (20 ml) and to the solution was carefully added trifluoroacetic acid (20 ml). The solution was left stirring at room temperature for 24 h and the solvents then removed under reduced pressure. Dichloromethane (40 ml) was then added and evaporated off, twice, followed by two similar treatments with THF. The solid residue was taken up in a minimum of DMF and THF added dropwise, until the solution just turned cloudy. It was left to stand overnight. The crystalline powder was collected, washed with THF (2×20 ml) and dried to afford a yellow powder.

RF 1202:

Compound 15 (1.0 mmol) was dissolved in water (60 ml) and Gadolium (III) acetate (1.0 mmol) was added slowly. During the addition the pH was maintained at 7.4 with sodium hydroxide. After addition the mixture was refluxed overnight. For desalting the mixture was applied to a C18-silicagel column that was rinsed with distilled water. Solvents were removed in vacuo and the product was obtained as a white solid. Identity of the product was confirmed by mass spectrometry.

### Example 15

### Preparation of RF 1203

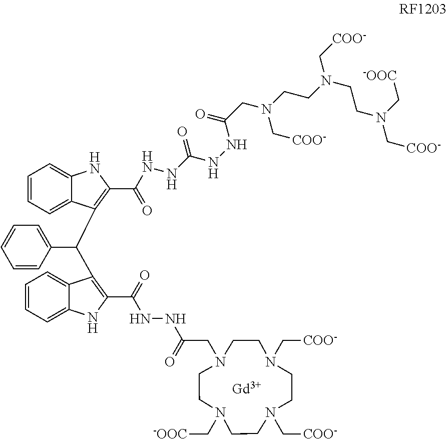

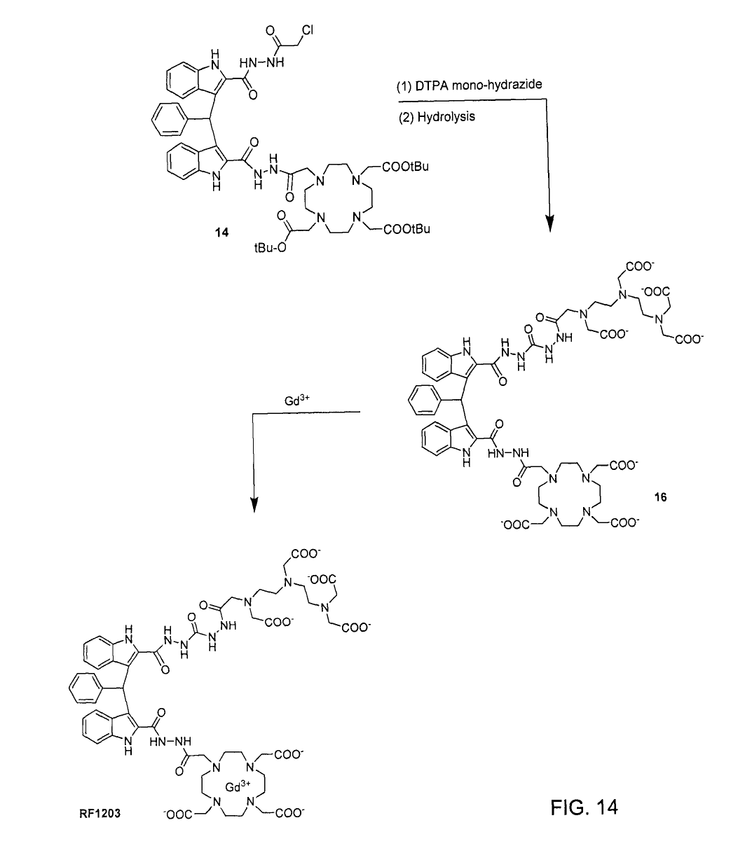

The RF 1203 compound was prepared following the process illustrated in FIG. 14.

RF 1203 Ligand (Compound 16):

To a mixture of compound 14 (2.13 g, 2 mmol) and triethylamine (0.20 g, 2 mmol)) in acetonitrile (40 ml) stirred under nitrogen, was added DTPA-mono-hydrazide (0.89 g, 2.2 mmol) (as discussed in Jolley, C., Appl. Radiat. Isot., 1996, 47(7), 623-626). The mixture was heated to a gentle reflux for 48 h. The reaction mixture was then cooled, filtered and the filtrate evaporated to dryness under reduced pressure to leave a yellow gum. The product was dissolved in a minimum volume of 10% NaHCO₃ solution and then chromatographed through a C18-silicagel column, using 10% acetonitrile-water as eluant, to yield a yellow solid. It was taken in THF (20 ml) and to the solution was carefully added trifluoroacetic acid (20 ml). The solution was left stirring at room temperature for 24 h and the solvents then removed under reduced pressure. Dichloromethane (40 ml) was then added and evaporated off, twice, followed by two similar treatments with THF. The solid residue was taken up in a minimum of DMF and THF added dropwise, until the solution just turned cloudy. It was left to stand overnight. The crystalline powder was collected, washed with THF (2×20 ml) and dried to afford a yellow powder.

RF 1203:

Compound 16 (1.0 mmol) was dissolved in water (60 ml) and Gadolium (III) acetate (1.0 mmol) was added slowly. During the addition the pH was maintained at 7.4 with sodium hydroxide. After addition the mixture was refluxed overnight. For desalting the mixture was applied on a C18-silicagel column that was rinsed with distilled water. Solvents were removed in vacuo and product was obtained as a white solid. Identity of the product was confirmed by mass spectrometry.

### Example 13

### MRI Analysis of Contrast Agents In Vivo

### Methods and Materials

Contrast agents were prepared following the methods outlined in Examples 1-11 above. Rats (250-300 g—obtained from where?) were used for the following studies detailed below.

All treatment and testing was conducted during the light hours. Animals were housed and tested in compliance with the guidelines described in the Guide to the Care and Use of Experimental Animals (Canadian Council on Animal Care, 1984; 1993). The McMaster University Committee for Animal Welfare approved all protocols.

### MRI in Rats

Rats were anesthetized by administration of isoflurane (5% induction, 1-2% maintenance) and positioned in an animal holder. The blood pressure and heart rate of the rats were monitored prior to ligation to provide a baseline condition. The rats were subjected to ligation of the left main coronary artery under aseptic conditions. During the ligation, the left thoracic cage of the rat, at the 4th intercostal space was opened to expose the heart muscle. The pericardial sac was cut open and the left main coronary artery was ligated at 2-4 mm from its origin using 6-0 prolene for a period of 2 h. The ligature was then removed to allow for the reperfusion of the infarcted myocardial tissue. Following the ligation procedure, bupivacaine and Cicatrin were applied to the incision. The incision was closed in layers, and ketoprofen (5 mg/kg) was injected subcutaneously to treat inflammation.

The contrast agent was the intravenously injected 4 hours following the start of myocardial reperfusion. The blood pressure and heart rate were monitored during the ligation procedure and the administration of the contrast agent. The body temperature of the rats was monitored with a rectal probe and maintained at the physiological level by the circulating warm water. The rats were individually housed following their recovery from the anesthesia.

Between about 4 to 16 hours following the administration of the contrast agent, the rats were anesthetized by an intramuscular injection of thiobutabarbital (100 mg/kg) and transported to the Life Sciences Centre for MRI imaging at the University of British Columbia. Animals were then positioned inside a 7 Tesla magnet imaging machine. A Volume quadrature resonantor was used for spin excitation and signal reception. Images of the entire heart were taken with an in-plane resolution of 234×234 microns. A 1 mm slice thickness was acquired using a multi-slice True-FISP pulse sequence triggered to the EKG signal. The pulse sequence parameters were optimized during an initial scan in order to achieve the optimal contrast between the infracted and viable myocardium. The total imaging time, including animal preparation and positioning, was approximately 60 minutes.

The rats were then immediately euthanized by administration of an overdose of pentobarbital (>120 mg/kg). The chest wall of the rats was then opened and the heart was harvested for fixing and staining of necrotic tissue. MR images and tissue sample histology was compared.

### Results

RF 1002

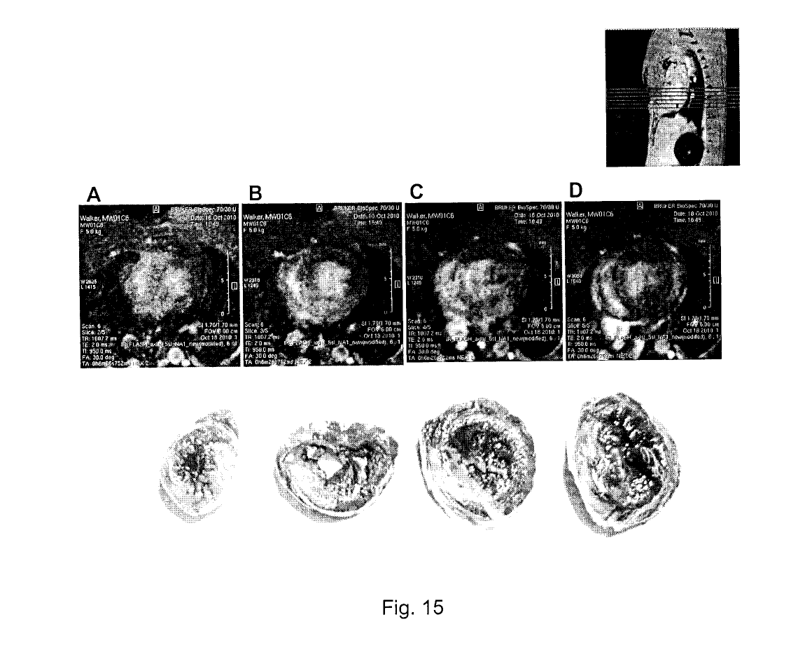

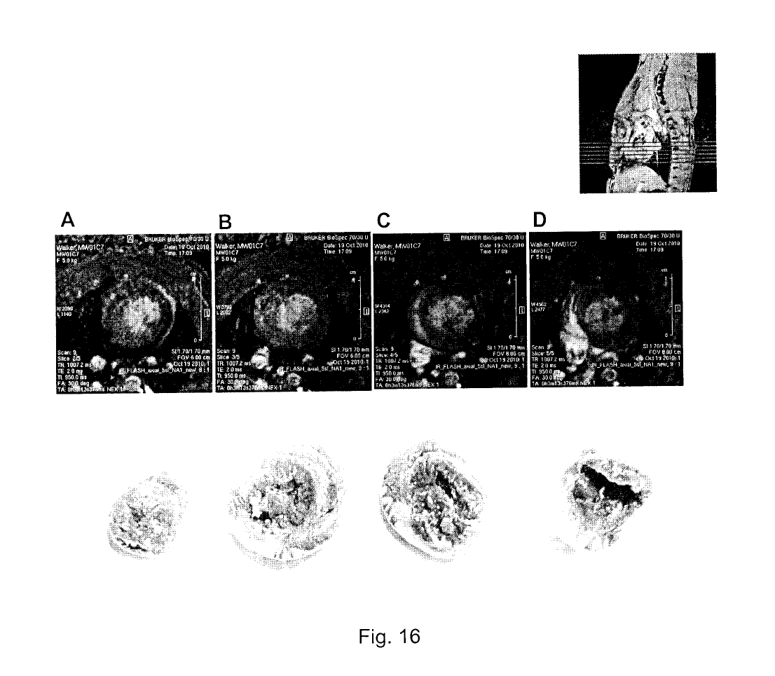

Contrast agent RF1002 was prepared following the method outlined in Example 1 above. Following the above-noted procedure, two groups of four rats were administered contrast agent RF1002, at a dose of 5 mg/kg and 40 mg/kg. The resultant MR image and corresponding tissue sample are shown in FIGS. 15 and 16 respectively.

The results illustrate that the necrotic tissue visible by staining of the tissue sample corresponds to the same region exhibiting contrast enhancement in the MR images. These result indicate that the contrast agent bound to necrotic tissue portion of the tissue sample. The visibility of contrast enhancement on the MRI is more defined at the higher dosage of contrast agent.

RF 1003

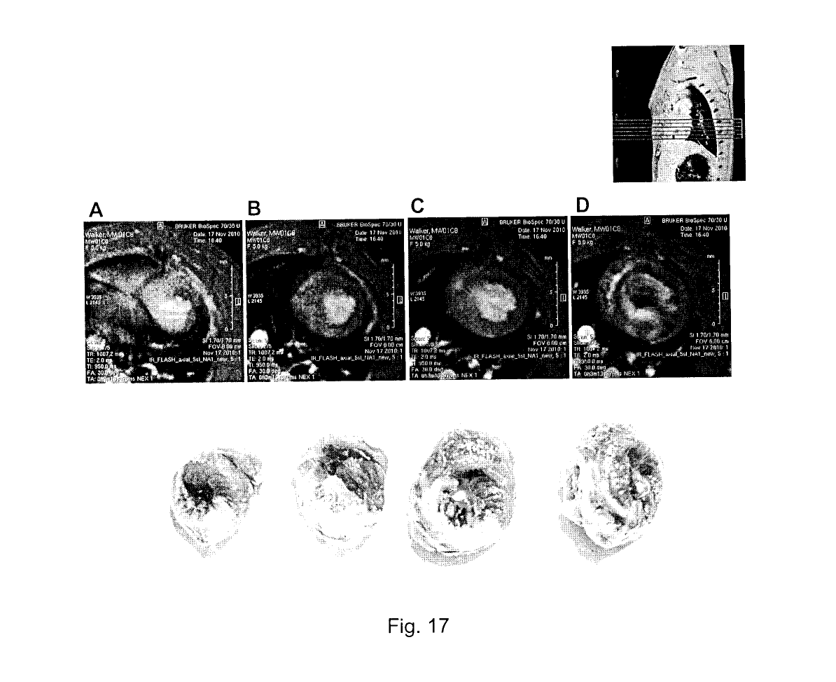

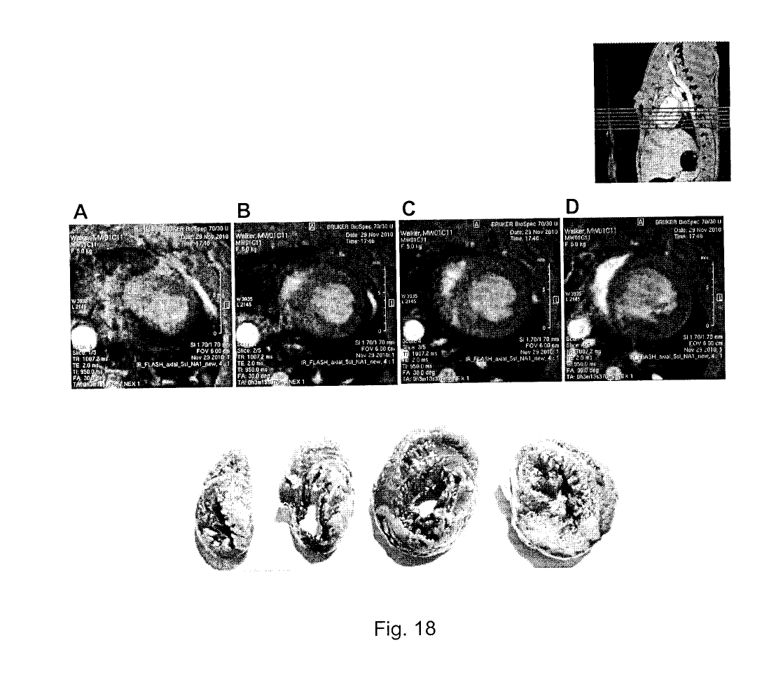

Contrast agent RF1003 was prepared following the method outlined in Example 2 above. Following the above-noted procedure, two groups of four rats were administered contrast agent RF1003, at a dose of 5 mg/kg and 40 mg/kg. The resultant MR image and corresponding tissue sample are shown in FIGS. 17 and 18 respectively.

The results illustrate that the necrotic tissue visible by staining of the tissue sample corresponds to the same region exhibiting contrast enhancement in the MR images. These result indicate that the contrast agent bound to necrotic tissue portion of the tissue sample. The visibility of contrast enhancement on the MRI is more defined at the higher dosage of contrast agent.

RF 1004

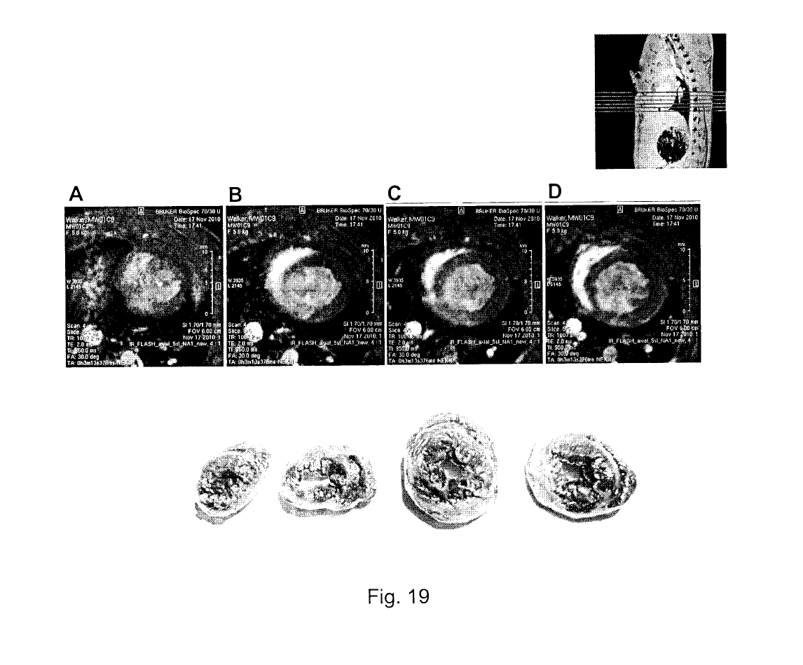

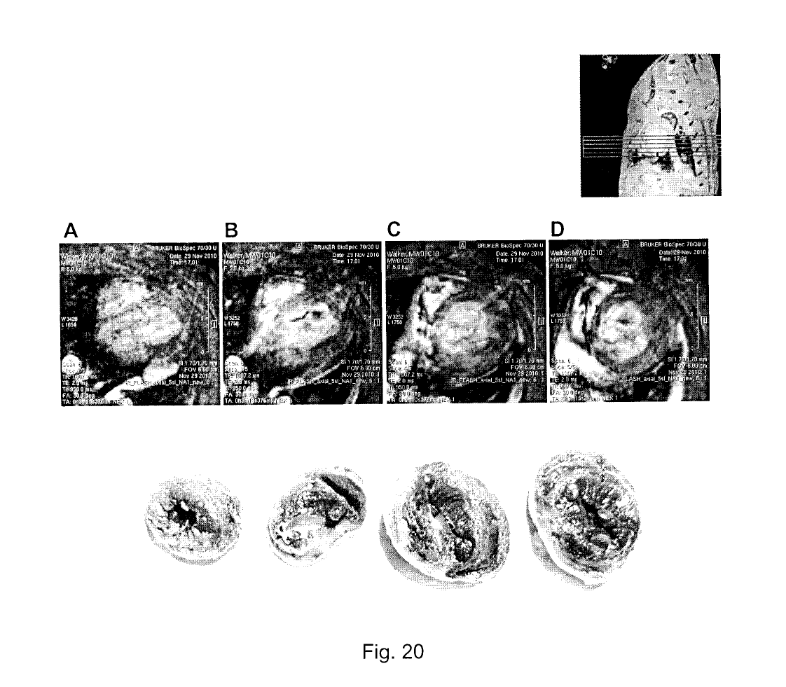

Contrast agent RF1004 was prepared following the method outlined in Example 3 above. Following the above-noted procedure, two groups of four rats were administered contrast agent RF1004, at a dose of 5 mg/kg and 40 mg/kg. The resultant MR image and corresponding tissue sample are shown in FIGS. 19 and 20 respectively.

The results illustrate that the necrotic tissue visible by staining of the tissue sample corresponds to the same region exhibiting contrast enhancement in the MR images. These result indicate that the contrast agent bound to necrotic tissue portion of the tissue sample. The visibility of contrast enhancement on the MRI is more defined at the higher dosage of contrast agent.

The above-described embodiments are intended to be examples only. Alterations, modifications and variations can be effected to the particular embodiments by those of skill in the art without departing from the scope, which is defined solely by the claims appended hereto.

What is claimed is:

1. A contrast agent for administration to a subject, the contrast agent comprising:
a targeting portion comprising an unchelated aminocarboxylate functional group,
a metal ion bound to a metal-complexable portion, and
a linker joining the targeting portion and the metal-complexable portion of the contrast agent,
wherein the portion that is not bound to a metal ion is for binding to necrotic tissue in the subject.

2. The contrast agent according to claim 1, wherein the metal-complexable portion of the contrast agent comprises an aminocarboxylate functional group.

3. The contrast agent according to claim 2, wherein the aminocarboxylate functional group of the contrast agent is a polyaminocarboxylate functional group.

4. The contrast agent according to any one of claims 1 to 3, wherein:
the targeting portion of the contrast agent is capable of complexing a metal ion;
only one metal ion is bound to the contrast agent;
the metal ion and the contrast agent are in a 1:1 molar ratio; and
one of the two portions of the contrast agent includes an unchelated aminocarboxylate functional group.

5. The contrast agent according to any one of claims 1 to 4, wherein the contrast agent comprises the structure X-L-Y*M, wherein X is the targeting portion, L is the linker, and Y*M is the metal ion (M) bound to the metal-complexable portion (Y) of the contrast agent and wherein only one metal ion is bound to the contrast agent and the metal ion and the contrast agent are in a 1:1 molar ratio.

6. The contrast agent according to any one of claims 1 to 5, wherein the aminocarboxyalte functional group is:

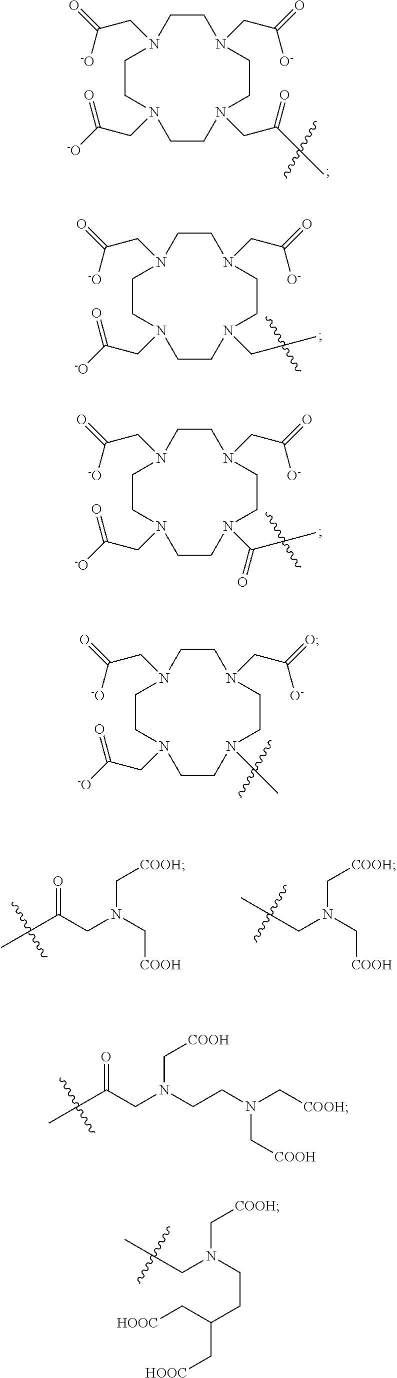

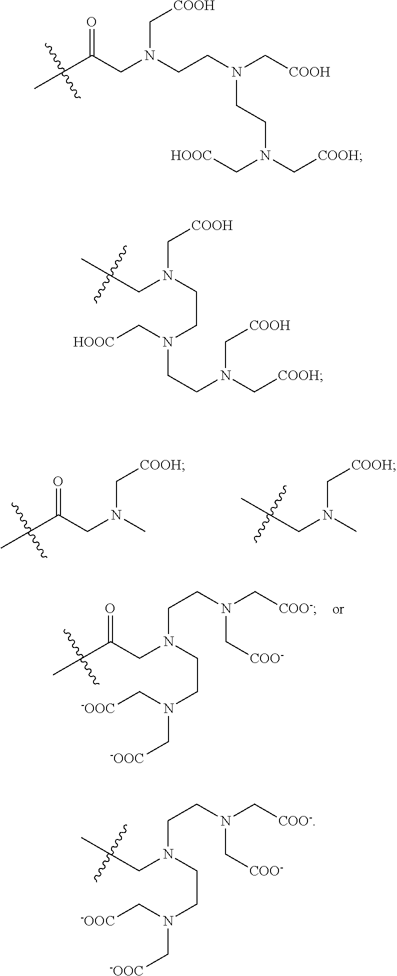

7. The contrast agent according to any one of claims 1 to 6, wherein the metal-complexable portion is:

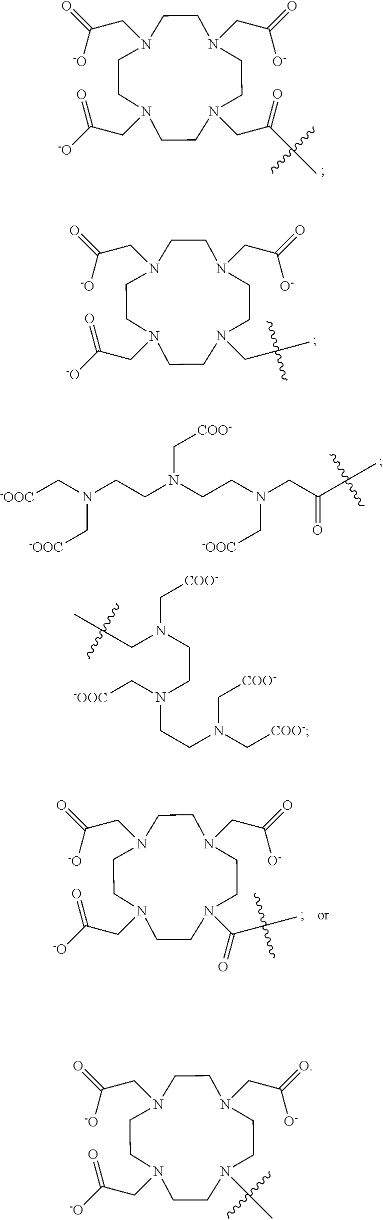

8. The contrast agent according to any one of claims 1 to 7, wherein the linker is: 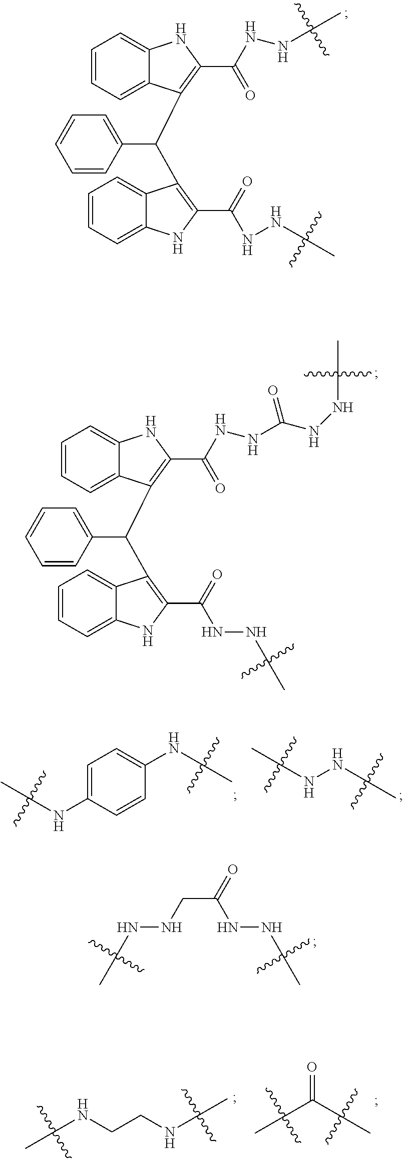
or a bond.

9. The contrast agent according to claim 1, wherein the metal ion is Gd³⁺ and contrast agent is:

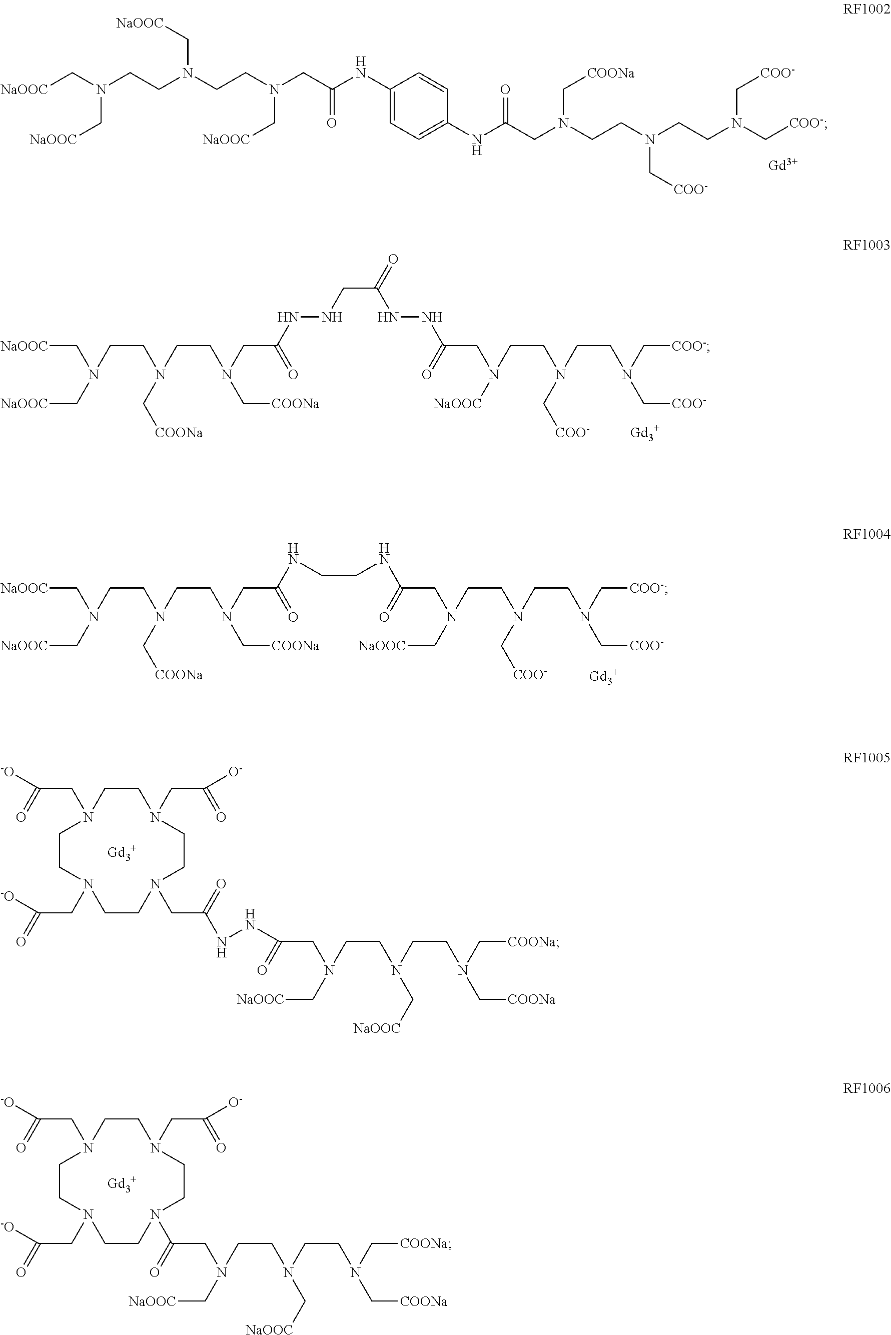

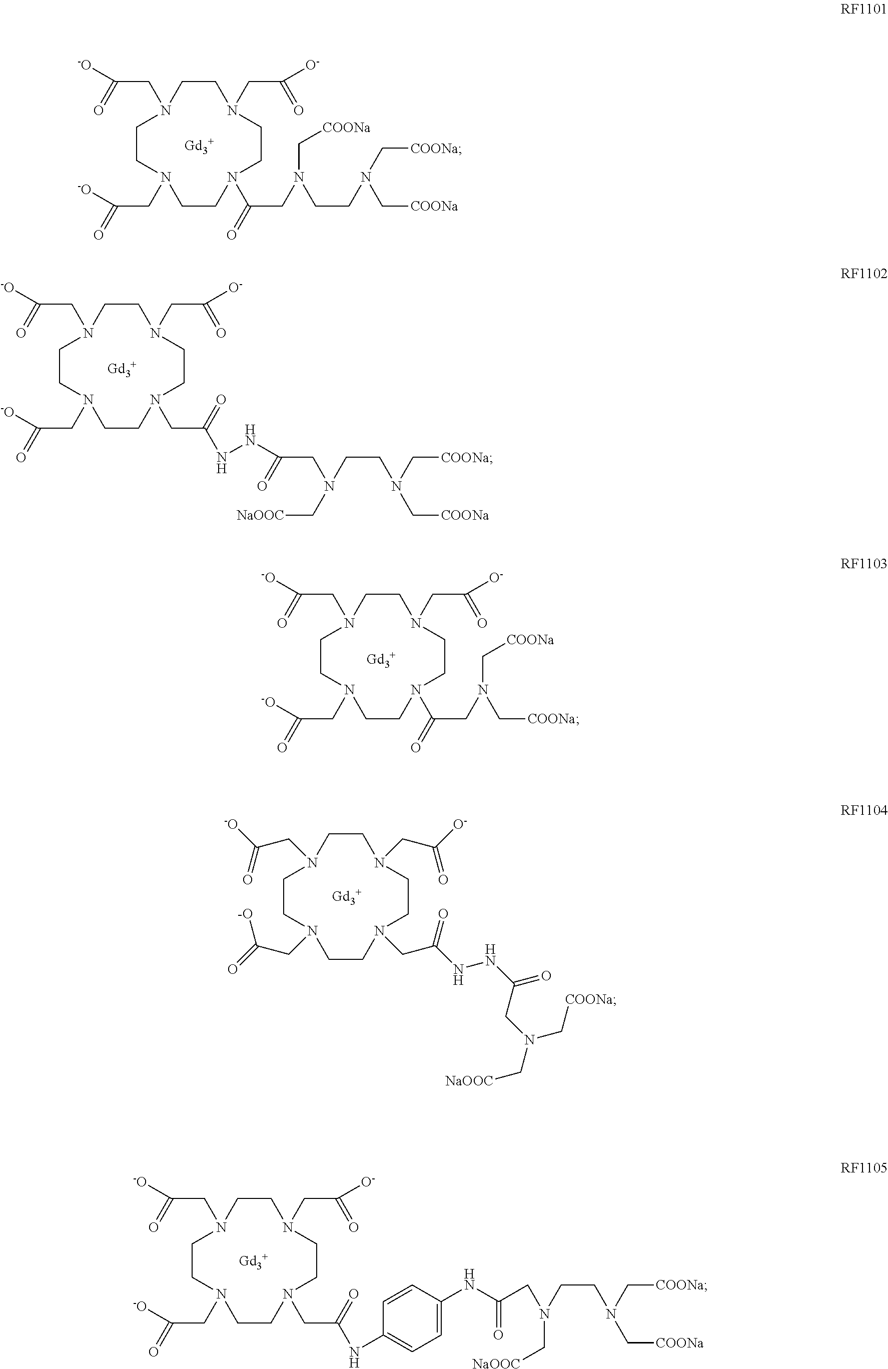

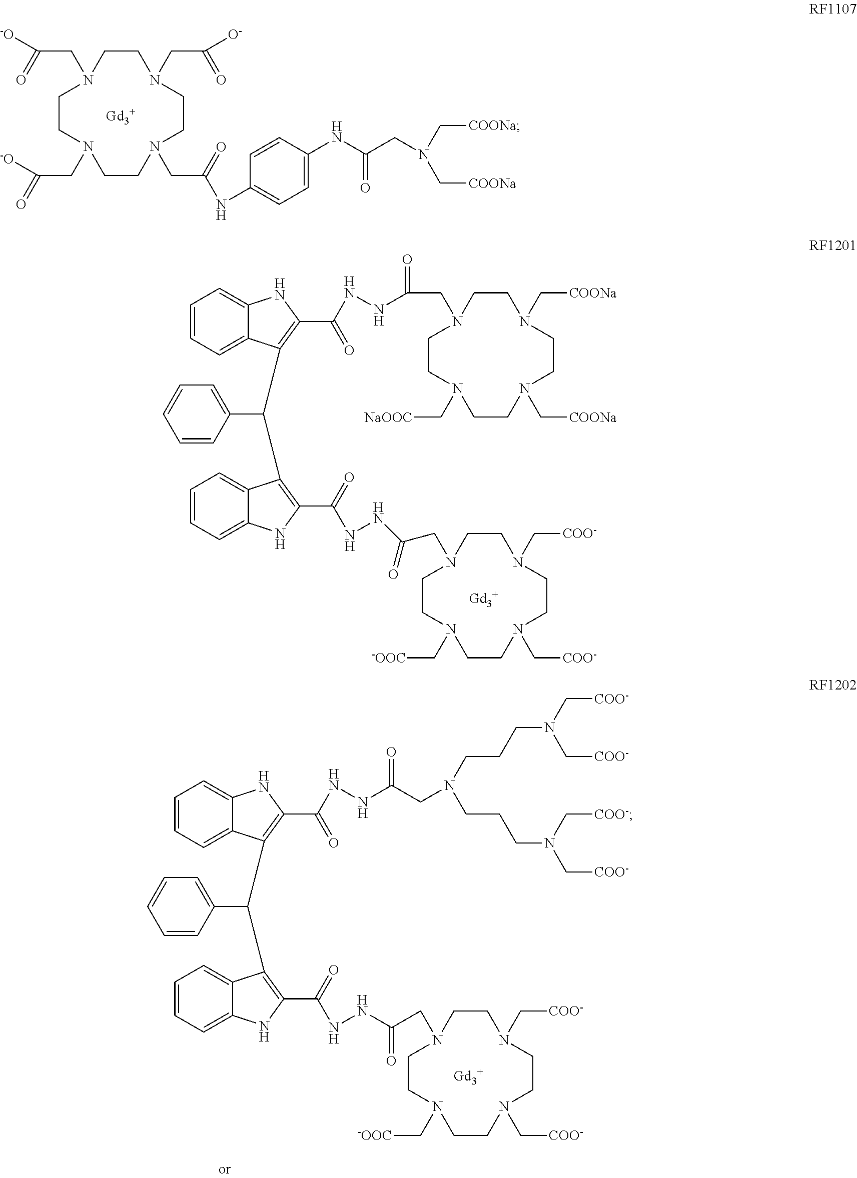

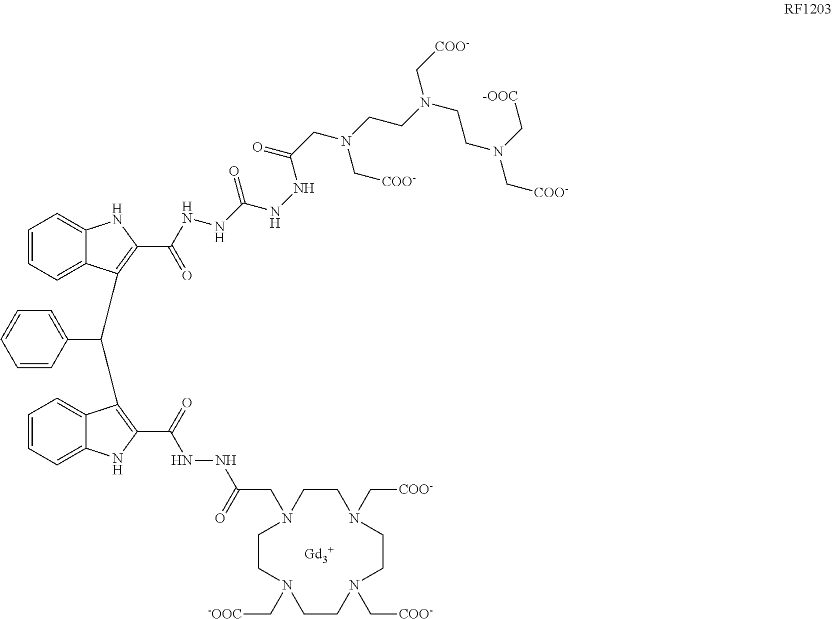

10. A composition comprising the contrast agent according to any one of claims 1 to 9, and a pharmaceutically acceptable diluent or carrier.

11. Use of the contrast agent according to any one of claims 1 to 9, or the composition according to claim 10, as a therapeutic agent, a diagnostic agent or both.

12. The use according to claim 11, wherein the contrast agent is for monitoring the effectiveness of an ongoing therapeutic treatment.

13. Use of the contrast agent according to any one of claims 1 to 9 in the manufacture of compounds and/or medicaments suitable for use in diagnostic imaging or imaging-aided applications.

14. The use according to claim 13 wherein the diagnostic imaging or imaging-aided application is magnetic resonance imaging (MRI), computed tomography (CT), single-photon emission computed tomography (SPECT), positron emission tomography (PET), MRI-aided application, CT-aided application, SPECT-aided application, or PET-aided application.
